(19) United States
(12) Patent Application Publication
(10) Pub. No.: US 2021/0031175 A1
(43) Pub. Date: Feb. 4, 2021

(54) A FAST BATCH PROCESS FOR PREPARING A ZEOLITIC MATERIAL HAVING FRAMEWORK TYPE CHA

(71) Applicants: BASF SE, Ludwigshafen am Rhein (DE); The University of Tokyo, Tokyo (JP)

(72) Inventors: Andrei-Nicolae PARVULESCU, Ludwigshafen (DE); Ulrich MUELLER, Ludwigshafen (DE); Kenta IYOKI, Bunkyo-ku (JP); Watcharop CHAIKITTISILP, Bunkyo-ku (JP); Torn WAKIHARA, Bunkyo-ku (JP)

(73) Assignees: BASF SE, Ludwigshafen am Rhein (DE); The University of Tokyo, Tokyo (JP)

(21) Appl. No.: 16/766,944

(22) PCT Filed: Dec. 20, 2018
(86) PCT No.: PCT/EP2018/086184

§ 371 Date: May 26, 2020

(30) Foreign Application Priority Data
Dec. 21, 2017 (EP) 17209758.6

(51) Int. Cl. B01J 29/70; C01B 39/48; B01J 35/10; B01J 37/00; B01J 37/04; B01J 37/10; C07C 1/04; B01D 53/94; F01N 3/28; F01N 3/20; F01N 3/08

(52) U.S. Cl. CPC B01J 29/7015; F01N 2370/04; B01J 35/1023; B01J 35/1019; B01J 37/0018; B01J 37/04; B01J 37/10; B01J 37/0036; C07C 1/0435; B01D 53/9418; F01N 3/2803; F01N 3/2066; F01N 3/0842; C01P 2002/60; C01P 2006/12; C01P 2002/86; C01P 2002/72; C01P 2004/03; C07C 2529/70; B01D 2255/50; B01D 2255/9207; C01B 39/48

(57) ABSTRACT

A batch process for preparing a zeolitic material having framework type CHA and a framework structure comprising Si, Al, O, and H, comprising (i) providing a seeding material comprising a zeolitic material having framework type CHA and a framework structure comprising Si, Al, O, and H; (ii) preparing a mixture comprising a source of Si, a source of Al, a seeding material provided in (i), a CHA framework structure directing agent comprising a cycloalkylammonium compound, and water, wherein the cycloalkylammonium compound is a compound comprising a cation R¹R²R³R⁴N⁺ wherein R¹, R², R³ are, independently from one another, an alkyl residue having from 1 to 6 carbon atoms, and R⁴ is a 5- to 8-membered cycloalkyl residue, wherein in mixture, the molar ratio of water relative to Si comprised in the source of Si and in the seeding material, calculated as SiO₂, is in the range of from 5:1 to 15:1, wherein the mixture, the molar ratio of sodium, calculated as Na₂O, relative to Si comprised in the source of Si and in the seeding material, calculated as SiO₂, is in the range of from 0:1 to 0.1:1; (iii) heating the mixture prepared in (ii) in its liquid state to a temperature of the mixture in the range of from 50 to 90° C. and keeping the liquid mixture at a temperature in this range for 5 to 100 h; (iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

The present invention relates to an ultrafast batch process for preparing a zeolitic material having framework type CHA wherein a cycloalkylammonium containing structure directing agent is used for crystallizing the zeolitic material.

Molecular sieves are classified by the Structure Commission of the International Zeolite Association according to the rules of the IUPAC Commission on Zeolite Nomenclature. According to this classification, framework-type zeolites and other crystalline microporous molecular sieves, for which a structure has been established, are assigned a three letter code and are described in the Atlas of Zeolite Framework Types, 5th edition, Elsevier, London, England (2001).

Among said zeolitic materials, Chabazite is a well studied example, wherein it is the classical representative of the class of zeolitic materials having a CHA framework structure. Zeolitic materials belonging to the class of molecular sieves having the CHA-type framework structure are employed in a variety of applications, and in particular serve as heterogeneous catalysts in a wide range of reactions such as in methanol to olefin catalysis and selective catalytic reduction of nitrogen oxides NOₓ to name some two of the most important applications. Zeolitic materials of the CHA framework type are characterized by three-dimensional 8-membered-ring (8MR) pore/channel systems containing double-six-rings (D6R) and cages. Zeolitic materials having a CHA-type framework structure and in particular Chabazite with incorporated copper ions (Cu—CHA) are widely used as heterogeneous catalyst for the selective catalytic reduction (SCR) of NOₓ fractions in automotive emissions. Based on the small pore openings and the alignment of the copper ions in the CHA cages, these catalyst systems have a unique thermal stability, which tolerates temperatures higher than 700° C. in presence of H₂O.

For the industrial production of CHA, cost intensive 1-adamantyltrimethyl-ammoniumhydroxide among other expensive organotemplates are typically employed as structure directing agent in the synthetic procedures for their preparation. U.S. Pat. No. 4,544,538 for example relates to the production of SSZ-13 using 1N-alkyl-3-quinuclidinol, N,N,N-tetraalkyl-1-adamantammonium, or N,N,N-trialkyl-exo-aminonorbornane as the structure directing agent, the SSZ-13 zeolitic material having a CHA-type framework structure. Compared to this teaching, WO 2015/185625 A provides a significant improvement in that a batch process had been developed by the respective inventors according to which a cycloalkylammonium containing structure directing agent is used making it possible to dispense with said adamantyltrimethylammonium containing organotemplate. Having a look at the examples of WO 2015/185625 A, one notes that the crystallization times are at least seven hours, and if crystallinity values of above 90% are should be obtained, the crystallization times are at least 17 h, and up to even 30 h, excluding the time necessary for heating the synthesis mixture to the desired crystallization temperature which, according to these examples, is 170° C. The maximum crystallinity achieved is 92%.

Fast crystallization times of CHA type zeolites have been achieved in EP 3020688 A1 using adamantyltrimethylammonium containing organotemplates and temperatures near 200° C. As opposed to the present invention, the EP 3020688 A1 patent requires adamantyltrimethyl-ammonium structure directing agent. The adamantyltrimethylammonium structure directing agent is more reactive than the inventive cycloalkylammonium SDAs and delivers 33% crystallinity without hydrothermal treatment under diluted conditions after aging of the reaction media at 85° C. as shown in comparative example 7 of the present invention. The critical impact of aging time and the addition of seeds to the beginning of the aging process of the present invention as related by comparative example 6 is not apparent from EP 3020688 A1. Moreover, EP 3020688 A1 demonstrates in example 6 of said invention that aging is unnecessary to achieve crystallinity values of 77% within 10 minutes of heating when using the adamantyl based structure directing reagents. Furthermore, the temperature limitations of the inventive cycloalkylammonium structure directing agents related by comparative example 4 are also not taught by EP 3020688 A1. Therefore, EP 3020688 A1 is completely silent with regards to developing a fast process for the cheaper and less reactive cycloalkylammonium based structure directing agents related in the present invention since it does not relate the combination of features required to realize the present invention.

In particular for industrial-scale batch processes for preparing zeolitic materials, it is generally desired to have crystallization times as short as possible, and, at the same time, achieving a crystallinity as high as possible.

Therefore, it was an object of the present invention to provide an improved batch process for preparing a zeolitic material having framework type CHA and a framework structure comprising Si, Al, O, and H, using a cycloalkylammonium compound as structure directing agent and being characterized by a very short crystallization time and, at the same time, a crystallinity of the obtained zeolitic material being at least comparable, preferably higher than those taught in the examples of WO 2015/185625 A.

Surprisingly, it was found that such a batch process wherein a cycloalkylammonium compound is employed as structure directing agent can be provided if it is designed so as to comprise a specific aging step.

Therefore, the present invention relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H;
(ii) preparing a mixture comprising a source of Si, a source of Al, a seeding material provided in (i), a CHA framework structure directing agent comprising a cycloalkylammonium compound, and water,
   wherein the cycloalkylammonium compound is a compound comprising a cation R¹R²R³R⁴N⁺ wherein R¹, R², R³ are, independently from one another, an alkyl residue having from 1 to 6 carbon atoms, and R⁴ is a 5- to 8-membered cycloalkyl residue, wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Regarding providing the seeding material according to (i), it has been found that it is especially preferred that prior to use, a given seeding material is milled. Therefore, it is preferred that providing the seeding material comprises preparing it by a method which comprises
(i.1) providing a zeolitic material having framework type CHA and a framework structure comprising Si, Al, O, and H;
(i.2) preparing a suspension comprising the zeolitic material provided in (i.1) and a liquid;
(i.3) milling the suspension prepared in (i.2).

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
(ii) preparing a mixture comprising a source of Si, a source of Al, a seeding material provided in (i), a CHA framework structure directing agent comprising a cycloalkylammonium compound, and water,
   wherein the cycloalkylammonium compound is a compound comprising a cation R¹R²R³R⁴N⁺ wherein R¹, R², R³ are, independently from one another, an alkyl residue having from 1 to 6 carbon atoms, and R⁴ is a 5- to 8-membered cycloalkyl residue,
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Regarding providing the zeolitic material having framework type CHA and a framework structure comprising Si, Al, O, and H according to (i.1), no specific restrictions exist. Generally, it is conceivable that, for example, it is prepared by a process as described in zeolitic material having framework type CHA and a framework structure comprising Si, Al, O, and H. Further, it may be preferred that it is prepared by a process according to the present invention. Further, it may be preferred that is prepared by a process as described in WO 2015/185625 A. Therefore, it is preferred that (i.1) comprises preparing a zeolitic material having a CHA-type framework structure comprising SiO₂ and Al₂O₃, wherein said process comprises the steps of
(a) providing a mixture comprising one or more sources for SiO₂, one or more sources for Al₂O₃, one or more tetraalkylammonium cation R¹R²R³R⁴N⁺-containing compounds, and one or more tetraalkylammonium cation R⁵R⁶R⁷R⁸N⁺-containing compounds as structure directing agent;
(b) crystallizing the obtained mixture, obtaining a zeolitic material having a CHA-type frame-work structure; wherein R¹, R², R³, R⁴, R⁵, R⁶, and R⁷ independently from one another stand for alkyl, and wherein R⁸ stands for cycloalkyl;

wherein R¹, R², R³, R⁴, R⁵, R⁶, and R⁷ independently from one another preferably stand for optionally substituted and/or optionally branched (C₁-C₆)alkyl, wherein R⁸ preferably stands for optionally heterocyclic and/or optionally substituted 5- to 8-membered cycloalkyl, wherein the crystallization is preferably conducted under solvothermal conditions, wherein the mixture provided in step (1) does not contain any substantial amount of a trimethyl benzyl ammonium containing compound, wherein the mixture provided in step (a) further comprises seed crystals. Thus, it is also preferred that the zeolitic material provided according to (i.1) is preferably the zeolitic material obtainable or obtained by said process, wherein the zeolitic material more preferably exhibits one or more of the following parameters:
an a first absorption band (B1) in the range of from 3,720 to 3,740 cm⁻¹; and a second absorption band (B2) in the range of from 1,850 to 1,890 cm⁻¹; wherein the ratio of the maximum absorbance of the first absorption band to the second absorption band B1:B2 is comprised in the range of from 0.5 to 1.55;
the particle size Dv10 of the zeolitic material is in the range of from 400 to 2,500 nm;
the particle size Dv50 of the zeolitic material is in the range of from 600 to 3,500 nm;
the particle size Dv90 of the zeolitic material is in the range of from 1,200 to 4,500 nm;
does not contain any substantial amount of the elements P and/or As;
the ²⁹Si MAS NMR of the zeolitic material comprises a first peak (P′1) in the range of from −102.0 to −106.0 ppm; and a second peak (P′2) in the range of from −108.0 to −112.5 ppm; wherein the integration of the first and second peaks in the ²⁹Si MAS NMR of the zeolitic material offers a ratio of the integration values P′1:P′2 comprised in the range of from 0.05 to 0.90;
the SiO₂:Al₂O₃ molar ratio of the framework structure of the zeolitic framework structure is in the range of from 4:1 to 200:1.

Regarding any further preferred characteristics of the process and the zeolitic material according to (i.1), reference is made to the respective disclosure of WO 2015/185625 A, the content of which is fully incorporated herein by reference.

As to preparing the suspension according to (i.2), it is preferred that said preparing comprises admixing the zeolitic material provided in (i.1) with a liquid, wherein the liquid preferably comprises water, wherein more preferably, from 95 to 100 weight-%, more preferably from 99 to 100 weight-% if the liquid consist of water, and wherein the weight ratio of the zeolitic material relative to the liquid is preferably in the range of from 1:10 to 1:50, preferably in the range of from 1:20 to 1:40, more preferably in the range of from 1:25 to 1:35. More preferred ranges are from 1:25 to 1:28 or from 1:28 to 1:32 or from 1:32 to 1:35.

Regarding milling the suspension according to (i.3), it preferably comprises bead-milling the suspension. The time for which the suspension is subjected to milling is preferably in the range of from 10 to 240 min, more preferably in the range of from 20 to 200 min, more preferably in the range of from 30 to 150 min. More preferred ranges are from 60 to 140 min, more preferably from 90 to 130 min. The beads which are preferably used have a diameter in the range of from 100 to 500 micrometer, more preferably in the range of from 200 to 400 micrometer, more preferably in the range of from 250 to 350 micrometer. The bead mill can be operated generally operated at any speed, wherein it is preferred that is operated at from 1,000 to 5,000 rpm, preferably from 2,000 to 4,000 rpm, more preferably from 2,500 to 3,500 rpm. The term “rpm” as used herein refers to “revolutions per minute”).

Preferably, step (i) may consist of (i.1), (i.2) and (i.3). According to this aspect of the present invention, the milled slurry which is obtained from (i.3) is employed as such and used as the seeding material.

According to further preferred aspect of the present invention, it may be preferred that providing the seeding material according to (i) further comprises, after (i.3),
(i.4) separating the zeolitic material comprised in the milled suspension obtained from (i.3) from the liquid.

According to this aspect, it is preferred that (i.4) comprises subjecting the milled suspension obtained from (i.3) to solid-liquid separation, preferably comprising one or more of filtration and centrifugation, obtaining a separated zeolitic material, and optionally drying the separated zeolitic material in gas atmosphere having a temperature in the range of from 20 to 100° C., wherein the gas atmosphere preferably comprises one or more of oxygen and nitrogen, thereby obtaining a dried seeding material. Further according to this aspect, it is preferred that the method for preparing the seeding material consists of (i.1), (i.2), (i.3), and (i.4).

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound, and water,
   wherein the cycloalkylammonium compound is a compound comprising a cation R¹R²R³R⁴N⁺ wherein R¹, R², R³ are, independently from one another, an alkyl residue having from 1 to 6 carbon atoms, and R⁴ is a 5- to 8-membered cycloalkyl residue,

wherein in mixture, the molar ratio of water relative to Si comprised in the source of Si and in the seeding material, calculated as SiO₂, is in the range of from 5:1 to 15:1,

wherein the mixture, the molar ratio of sodium, calculated as Na₂O, relative to Si comprised in the source of Si and in the seeding material, calculated as SiO₂, is in the range of from 0:1 to 0.1:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

The seeding material which is employed is preferably used in an amount so that in the mixture prepared in (ii), the weight ratio of the seeding material, relative to the Si comprised in the source of Si, calculated as SiO₂, is in the range of from 0.025:1 to 0.15:1, preferably in the range of from 0.030:1 to 0.13:1, more preferably in the range of from 0.035:1 to 0.11:1.

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) preferably separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound, and water,
   wherein the cycloalkylammonium compound is a compound comprising a cation R¹R²R³R⁴N⁺ wherein R¹, R², R³ are, independently from one another, an alkyl residue having from 1 to 6 carbon atoms, and R⁴ is a 5- to 8-membered cycloalkyl residue,
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
   wherein in the mixture, the weight ratio of the seeding material, relative to the Si comprised in the source of Si, calculated as SiO₂, is in the range of from 0.035:1 to 0.11:1;
(iii) heating the mixture prepared in (ii) in its liquid state to a temperature of the mixture in the range of from 50 to 90° C. and keeping the liquid mixture at a temperature in this range for 5 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Regarding the amount of the seeding material, it is preferred, according to a first aspect, that in the mixture prepared in (ii), the weight ratio of the seeding material, relative to the Si comprised in the source of Si, calculated as SiO₂, is in the range of from 0.025:1 to 0.060:1, preferably in the range of from 0.030:1 to 0.055:1, more preferably in the range of from 0.035:1 to 0.045:1. Further, according to a second aspect, it is preferred that in the mixture prepared in (ii), the weight ratio of the seeding material, relative to the Si comprised in the source of Si, calculated as SiO₂, is in the range of from 0.085:1 to 0.15:1, preferably in the range of from 0.090:1 to 0.13:1, more preferably in the range of from 0.095:1 to 0.11:1.

As to the CHA framework structure directing agent according to (ii), it is preferred that R¹, R², R³ are, independently from one another, an alkyl residue having from 1 to 5 carbon atoms, preferably from 1 to 4 carbon atoms, more preferably from 1 to 3 carbon atoms, more preferably 1 or 2 carbons, more preferably 1 carbon atom, and wherein R⁴ is a 5- to 7-membered cycloalkyl residue, preferably a 5- or 6-membered cycloalkyl residue, more preferably a 6-membered cycloalkyl residue. More preferably, R¹, R², R³ are methyl and R⁴ is cyclohexyl. Preferably, the cycloalkylammonium compound comprised in the CHA framework structure directing agent according to (ii) comprises, preferably is an ammonium salt, preferably one or more of a halide, a sulfate, a nitrate, an acetate, and a hydroxide, more preferably one or more of a chloride, a bromide, a sulfate, a nitrate, an acetate, and a hydroxide, more preferably one or more of a chloride, a bromide, and a hydroxide, more preferably a hydroxide. More preferably, the cycloalkylammonium compound comprised in the CHA framework structure directing agent according to (ii) comprises, preferably is N,N,N-trimethyl-cyclohexylammonium hydroxide.

Regarding the CHA framework structure directing agent, and according to a first aspect of the present invention, it is possible that preferably from 99 to 100 mol-%, more preferably from 99.5 to 100 mol-%, more preferably from 99.9 to 100 mol-% of the CHA framework structure directing agent consist of N,N,N-trimethyl-cyclohexylammonium hydroxide, wherein more preferably, the CHA framework structure directing agent is free of N,N,N-trimethyl-1-adamantylammonium hydroxide, preferably free of an N,N,N-trimethyl-1-adamantylammonium comprising compound, more preferably free of an adamantylammonium comprising compound.

Regarding the CHA framework structure directing agent, and according to a first aspect of the present, it is preferred that the CHA framework structure directing agent according to (ii) further comprises a tetraalkylammonium compound comprising a cation R⁵R⁶R⁷R⁸N⁺ wherein R⁵, R⁶, R⁷, R⁸ are, independently from one another, an optionally substituted alkyl residue having from 1 to 4 carbon atoms, preferably from 1 to 3 carbon atoms, more preferably 1 or 2 carbons, wherein more preferably, R⁵, R⁶, R⁷, R⁸ are methyl, wherein the substituent is preferably one or more of chloro and hydroxyl, more preferably hydroxyl. Thus, in case one of the residues is substituted, it may be preferred that the tetraalkylammonium compound comprises, preferably is, a 2-hydroxyethyl-trimethylammonium compound. Preferably, the tetraalkylammonium compound comprised in the CHA framework structure directing agent according to (ii) comprises, preferably is an ammonium salt, preferably one or more of a halide, a sulfate, a nitrate, an acetate, and a hydroxide, more preferably one or more of a chloride, a bromide, a sulfate, a nitrate, an acetate, and a hydroxide, more preferably one or more of a chloride, a bromide, and a hydroxide, more preferably a hydroxide. More preferably, the tetraalkylammonium compound comprised in the CHA framework structure directing agent according to (ii) comprises, preferably is tetramethylammonium hydroxide.

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) preferably separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound and a tetraalkylammonium compound, and water,
   wherein the cycloalkylammonium compound comprises, preferably is, N,N,N-trimethyl-cyclohexylammonium hydroxide, and the tetraalkylammonium compound comprises, preferably is, tetramethylammonium hydroxide;
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
   wherein in the mixture, the weight ratio of the seeding material, relative to the Si comprised in the source of Si, calculated as SiO₂, is in the range of from 0.035:1 to 0.11:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Preferably, in the CHA framework structure directing agent according to (ii), the molar ratio of the cycloalkylammonium compound relative to the tetraalkylammonium compound is in the range of from 1:1 to 5.5:1, more preferably in the range of from 1.1:1 to 4:1, more preferably in the range of from 1.3:1 to 3:1, more preferably in the range of from 1.5:1 to 2.0:1. Preferred ranges are, from example, from 1.5:1 to 1.9:1, or from 1.5.1 to 1.8:1, or from 1.5:1 to 1.7:1, or from 1.5:1 to 1.6:1.

Regarding the CHA framework structure directing agent, and according to said second aspect of the present invention, it is preferred that from 99 to 100 mol-%, more preferably from 99.5 to 100 mol-%, more preferably from 99.9 to 100 mol-% of the CHA framework structure directing agent consist of the cycloalkylammonium compound and the tetraalkylammonium compound, wherein more preferably, the CHA framework structure directing agent is free of N,N,N-trimethyl-1-adamantylammonium hydroxide, preferably free of an N,N,N-trimethyl-1-adamantylammonium comprising compound, more preferably free of an adamantylammonium comprising compound.

Preferably, in the mixture prepared in (ii), the molar ratio of the CHA framework structure directing agent relative to Si comprised in the source of Si and in the seeding material, calculated as SiO₂, is in the range of from 0.20:1 to 0.30:1, more preferably in the range of from 0.22:1 to 0.29:1, more preferably in the range of from 0.25:1 to 0.28:1.

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) preferably separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound and a tetraalkylammonium compound, and water,
   wherein the cycloalkylammonium compound comprises, preferably is, N,N,N-trimethyl-cyclohexylammonium hydroxide, and the tetraalkylammonium compound comprises, preferably is, tetramethylammonium hydroxide;
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
   wherein in the mixture, the weight ratio of the seeding material, relative to the Si comprised in the source of Si, calculated as SiO₂, is in the range of from 0.035:1 to 0.11:1;
   wherein in the mixture, the molar ratio of the CHA framework structure directing agent relative to Si comprised in the source of Si and in the seeding material, calculated as SiO₂, is in the range of from 0.25:1 to 0.28:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Preferably, the amount of water comprised in the mixture prepared in (ii) is comparatively low. More preferably, in mixture prepared in (ii), the molar ratio of water relative to Si comprised in the source of Si and in the seeding material (Si comprised in the source of Si plus Si comprised in the seeding material), calculated as SiO₂, is in the range of from 7:1 to 15:1, preferably in the range of from 9:1 to 12:1.

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) preferably separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound and a tetraalkylammonium compound, and water,
   wherein the cycloalkylammonium compound comprises, preferably is, N,N,N-trimethyl-cyclohexylammonium hydroxide, and the tetraalkylammonium compound comprises, preferably is, tetramethylammonium hydroxide;
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
   wherein in the mixture, the weight ratio of the seeding material, relative to the Si comprised in the source of Si, calculated as SiO₂, is in the range of from 0.035:1 to 0.11:1;
   wherein in the mixture, the molar ratio of the CHA framework structure directing agent relative to Si comprised in the source of Si and in the seeding material, calculated as SiO₂, is in the range of from 0.25:1 to 0.28:1;
   wherein in mixture, the molar ratio of water relative to Si comprised in the source of Si and in the seeding material is in the range of from 9:1 to 12:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Regarding the source of silicon according to (ii), every suitable source can be employed. Preferably, the source of Si comprises, more preferably is, one or more of a silica and a silicate, preferably one or more of a fumed silica, a silica sol, an amorphous silica, a silica gel, a silicic acid, a silic acid ester, colloidal silica, a tetraalkoxysilane, a disilicate, and a sesquisilcate, more preferably one or more of a fumed silica, a silica hydrosol, a silica gel, a silicic acid, a silicic acid ester, a colloidal silica, and a tetraalkoxysilane, more preferably one or more of a fumed silica, a silica hydrosol, a silica gel, and a colloidal silica, more preferably one or more of a fumed silica, a silica gel, and a colloidal silica. More preferably, the source of Si comprises, more preferably is, a colloidal silica.

In particular regarding the colloidal silica, it may be possible that an activated colloidal silica as described in US 20170113941 A is employed, i.e. a modified colloidal silica sol which has been modified with a metal compound and in the presence of a structure directing agent wherein the structure directing agent is preferably a structure directing agent as described above comprising a cycloalkylammonium compound, more preferably a structure directing agent as described above comprising a cycloalkylammonium compound and a tetraalkylammonium compound, and wherein the metal may be one of the metals described in US 20170113941 A wherein it may be preferred that the metal does not comprise sodium, more preferably does not comprise an alkali metal.

Regarding the source of aluminum according to (ii), every suitable source can be employed. Preferably, the source of Al comprises, more preferably is, one or more of an alumina, an aluminate, and an aluminum salt, preferably one or more of an alumina and an aluminum salt, more preferably one or more of an alumina, a tri(C1-C5)alkoxide, an AlO(OH), an Al(OH)₃, an aluminum halide wherein the aluminum halide is preferably one or more of an aluminum fluoride, an aluminum chloride and an aluminum bromide, an aluminum sulfate, an aluminum phosphate, and an aluminum fluorosilicate, more preferably one or more of an AlO(OH) and an Al(OH)₃. More preferably, the source of source of Al comprises, more preferably is, an Al(OH)₃, more preferably a crystalline Al(OH)₃, more preferably gibbsite.

Preferably, in the mixture prepared in (ii), the molar ratio of the source of Al, calculated as Al₂O₃, relative to Si comprised in the source of Si and in the seeding material, calculated as SiO₂, is in the range of from 0.001:1 to 0.5:1, more preferably in the range of from 0.01:1 to 0.1:1, preferably in the range of from 0.02:1 to 0.05:1, more preferably in the range of from 0.03:1 to 0.04:1.

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) preferably separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound and a tetraalkylammonium compound, and water,
   wherein the cycloalkylammonium compound comprises, preferably is, N,N,N-trimethyl-cyclohexylammonium hydroxide, and the tetraalkylammonium compound comprises, preferably is, tetramethylammonium hydroxide;
   wherein the source if Si comprises, preferably is, a colloidal silica;
   wherein the source of Al comprises, preferably is, an Al(OH)₃, preferably a crystalline Al(OH)₃;
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
   wherein in the mixture, the weight ratio of the seeding material, relative to the Si comprised in the source of Si, calculated as SiO₂, is in the range of from 0.035:1 to 0.11:1;
   wherein in the mixture, the molar ratio of the CHA framework structure directing agent relative to Si comprised in the source of Si and in the seeding material, calculated as SiO₂, is in the range of from 0.25:1 to 0.28:1;
   wherein in mixture, the molar ratio of water relative to Si comprised in the source of Si and in the seeding material is in the range of from 9:1 to 12:1;
   wherein in the mixture, the molar ratio of the source of Al, calculated as Al₂O₃, relative to Si comprised in the source of Si and in the seeding material, calculated as SiO₂, is in the range of from 0.03:1 to 0.04:1;
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Preferably according to the present invention, from 95 to 100 weight-%, preferably from 98 to 100 weight-%, more preferably from 99 to 100 weight-% of the mixture prepared in (ii) consist of the source of Si, the source of Al, the seeding material provided in (i), the CHA framework structure directing agent comprising a cycloalkylammonium compound, and the water. More preferably according to the present invention, from 99.5 to 100 weight-%, more preferably from 99.9 to 100 weight-%, more preferably from 99.9 to 100 weight-% of the mixture prepared in (ii) consist of the source of Si, the source of Al, the seeding material provided in (i), the CHA framework structure directing agent comprising a cycloalkylammonium compound, and the water. Preferably according to the present invention, in the mixture prepared in (ii), the molar ratio of phosphorus, calculated as elemental P, relative to Si comprised in the source of Si and in the seeding material, calculated as SiO₂, is in the range of from 0:1 to 0.001:1.

Preferably, according to (ii), preparing the mixture comprises
(ii.1) preparing a mixture comprising the source of Si, the source of Al, the CHA framework structure directing agent comprising a cycloalkylammonium compound, and water;
(ii.2) adding the seeding material to the mixture prepared in (ii.1), obtaining the mixture to be subjected to (iii).

Regarding this preferred preparation of the mixture, it is further preferred that
(ii.1) comprises
(ii.1.1) preparing a mixture comprising the source of Al and the CHA framework structure directing agent comprising a cycloalkylammonium compound;
(ii.1.2) agitating the mixture prepared in (ii.1.1) at a temperature of the mixture in the range of from 10 to 50° C. for 5 to 60 min;
(ii.1.3) adding the source of Si to the mixture obtained from (ii.1.2);
(ii.1.4) agitating the mixture prepared in (ii.1.3) at a temperature of the mixture in the range of from 10 to 50° C. for 1 to 30 min, obtaining the mixture to be subjected to (iii).

Therefore, the present invention preferably relates to a batch process for preparing a zeolitic material having framework type CHA and a framework structure comprising Si, Al, O, and H, comprising
(i) providing a seeding material comprising a zeolitic material having framework type CHA and a framework structure comprising Si, Al, O, and H, comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2);
   (i.4) preferably separating the zeolitic material comprised in the milled suspension obtained from (i.3) from the liquid;
(ii) preparing a mixture comprising a source of Si, a source of Al, a seeding material provided in (i), a CHA framework structure directing agent comprising a cycloalkylammonium compound and a tetraalkylammonium compound, and water,
   wherein the cycloalkylammonium compound comprises, preferably is, N,N,N-trimethyl-cyclohexylammonium hydroxide, and the tetraalkylammonium compound comprises, preferably is, tetramethylammonium hydroxide;
   wherein the source if Si comprises, preferably is, a colloidal silica;
   wherein the source of Al comprises, preferably is, an Al(OH)₃, preferably a crystalline Al(OH)₃;
   wherein in mixture, the molar ratio of water relative to Si comprised in the source of Si and in the seeding material, calculated as SiO₂, is in the range of from 5:1 to 15:1,
   wherein the mixture, the molar ratio of sodium, calculated as Na₂O, relative to Si comprised in the source of Si and in the seeding material, calculated as SiO₂, is in the range of from 0:1 to 0.1:1;
   wherein in the mixture, the weight ratio of the seeding material, relative to the Si comprised in the source of Si, calculated as SiO₂, is in the range of from 0.035:1 to 0.11:1;
   wherein in the mixture, the molar ratio of the CHA framework structure directing agent relative to Si comprised in the source of Si and in the seeding material, calculated as SiO₂, is in the range of from 0.25:1 to 0.28:1;
   wherein in mixture, the molar ratio of water relative to Si comprised in the source of Si and in the seeding material is in the range of from 9:1 to 12:1;
   wherein in the mixture, the molar ratio of the source of Al, calculated as Al₂O₃, relative to Si comprised in the source of Si and in the seeding material, calculated as 510₂, is in the range of from 0.03:1 to 0.04:1;
   wherein (ii) comprises
   (ii.1) preparing a mixture comprising the source of Si, the source of Al, the CHA framework structure directing agent comprising a cycloalkylammonium compound, and water;
   (ii.2) adding the seeding material to the mixture prepared in (ii.1), obtaining the mixture to be subjected to (iii); wherein (ii.2) preferably comprises
      (ii.1.1) preparing a mixture comprising the source of Al and the CHA framework structure directing agent comprising a cycloalkylammonium compound;
      (ii.1.2) agitating the mixture prepared in (ii.1.1) at a temperature of the mixture in the range of from 10 to 50° C. for 5 to 60 min;
      (ii.1.3) adding the source of Si to the mixture obtained from (ii.1.2);
      (ii.1.4) agitating the mixture prepared in (ii.1.3) at a temperature of the mixture in the range of from 10 to 50° C. for 1 to 30 min, obtaining the mixture to be subjected to (iii);
(iii) heating the mixture prepared in (ii) in its liquid state to a temperature of the mixture in the range of from 50 to 90° C. and keeping the liquid mixture at a temperature in this range for 12 to 100 h;
(iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.

Preferably, according to (ii.1.2), the mixture is agitated at a temperature of the mixture in the range of from 15 to 40° C., preferably in the range of from 20 to 30° C. Further preferably, according to (ii.1.2), the mixture is agitated for 10 to 50 min, more preferably for 20 to 40 min. Further preferably, according to (ii.1.2), the mixture is agitated at a pressure in the range of from 0.7 to 2 bar(abs), more preferably in the range of from 0.8 to 1.5 bar(abs), more preferably in the range of from 0.9 to 1.1 bar(abs). Agitating according to (ii.1.2) preferably comprises mechanically agitating the mixture, more preferably stirring the mixture, preferably at from 100 to 1,000 rpm, more preferably from 200 to 750 rpm, more preferably from 400 to 600 rpm.

Preferably, according to (ii.1.4), the mixture is agitated at a temperature of the mixture in the range of from 15 to 40° C., preferably in the range of from 20 to 30° C. Further preferably, according to (ii.1.4), the mixture is agitated for 2 to 20 min, more preferably for 5 to 15 min. Further preferably, according to (ii.1.4), the mixture is agitated at a pressure in the range of from 0.7 to 2 bar(abs), more preferably in the range of from 0.8 to 1.5 bar(abs), more preferably in the range of from 0.9 to 1.1 bar(abs). Agitating according to (ii.1.4) preferably comprises mechanically agitating the mixture, more preferably stirring the mixture, preferably stirring at from 100 to 1,000 rpm, more preferably from 200 to 750 rpm, more preferably from 400 to 600 rpm.

According to preferred aspect of the present invention regarding (ii.1), (ii.1) consists of (ii.1.1), (ii.1.2), (ii.1.3) and (ii.1.4). According to a further preferred aspect of the present invention regarding (ii), (ii) consists of (ii.1) and (ii.2).

Preferably, according to (iii), the mixture prepared (ii) is heated in its liquid state to a temperature of the mixture in the range of from 55 to 80° C., more preferably in the range of from 60 to 70° C. Further preferably, according to (iii), the liquid mixture is kept at the temperature for 5 to 80 h, more preferably for 20 to 50 h. Preferred ranges are, for example, from 20 to 30 h or from 25 to 35 h or from 30 to 40 h or from 35 to 45 h or from 40 to 50 h. Preferably, heating the mixture according to (iii), more preferably heating the mixture and keeping the mixture at the temperature is carried out at a pressure in the range of from 0.7 to 2 bar(abs), more preferably in the range of from 0.8 to 1.5 bar(abs), more preferably in the range of from 0.9 to 1.1 bar(abs). Preferably, according to (iii), the mixture is heated to the temperature at a temperature ramp in the range of from 0.2 to 5 K/min, preferably in the range of from 0.5 to 4 K/min more preferably in the range of from 1 to 3 K/min. During keeping the mixture at the temperature according to (iii), preferably during heating the mixture and keeping the mixture at the temperature according to (iii), the mixture is agitated wherein said agitating preferably comprises mechanically agitating the mixture, more preferably stirring the mixture. Stirring is preferably performed at from 100 to 1,000 rpm, more preferably from 200 to 750 rpm, more preferably from 400 to 600 rpm.

According to a preferred aspect of the present invention regarding (iii), (iii) consists of heating the mixture and keeping the mixture at the temperature.

Preferably, according to (iv), the mixture of (iii) is heated to a temperature of the mixture in the range of from 195 to 225° C., more preferably in the range 200 to 220° C. Preferred ranges are, for example, from 200 to 210° C. or from 205 to 215° C. or from 210 to 220° C. According to (iv), the mixture is preferably kept at the temperature for 0.75 to 7.5 h, more preferably for 1 to 5 h. Preferred ranges are, for example, from 1 to 3 h or from 2 to 4 h or from 3 to 5 h. Preferably, according to (iv), the mixture is heated to the temperature at a temperature ramp in the range of from 0.1 to 20 K/min, more preferably in the range of from 0.5 to 15 K/min, more preferably in the range of from 1 to 10 K/min. Preferred ranges are, for example, from 1 to 5 K/min or from 2 to 4 K/min or from 5 to 10 K/min or from 6 to 10 K/min or from 7 to 10 K/min or from 8 to 10 K/min. During heating the mixture or keeping the mixture at the temperature according to (iv), preferably during heating the mixture and keeping the mixture at the temperature according to (iv), the mixture is preferably agitated, more preferably mechanically agitated, wherein more preferably, the crystallization vessel is agitated. Agitating the crystallization vessel is, for example, performed by tumbling the crystallization vessel.

The crystallization according to (iv) can be carried out in every suitable batch crystallization vessel such as an autoclave or a sealable tubular reactor. Regarding preferred tubular reactors which may be used according to the process of the present invention and the respective process steps, particular reference is made to embodiments 62 to 71 hereinbelow. Preferably, the tubular reactor comprises a reaction tube and one or two sealing caps for sealing the reaction tube. For heating purposes, comprising heating the mixture and keeping the mixture at the temperature according to (iv), the tubular reactor can externally heated by a heating medium, wherein the heating medium may comprise a gaseous heating medium, a liquid heating medium, or a solid heating medium, preferably a gaseous heating medium or a liquid heating medium. Steam and electrical heating can also be performed. If the heating medium is a gaseous heating medium, which is preferably contained in a statically or a continuously operated oven, the gaseous heating medium is statically or continuously brought in contact with the reaction tube during heating and keeping at the temperature according to (iv). If the heating medium is a liquid heating medium, preferably comprising an oil, said heating medium more preferably being contained in a statically or a continuously operated bath, the liquid heating medium is statically or continuously brought in contact with the reaction tube during heating and keeping at the temperature according to (iv). The reaction tube is preferably made of material having a thermal diffusivity in the range of from 3×10⁻⁶ to 30×10⁻⁶ m²/s, more preferably in the range of from 5×10⁻⁶ to 25×10⁻⁶ m²/s. Preferably, the material may be stainless steel. Preferably, the ratio of the volume V/cm³ of the reaction tube to the outer surface area A/cm² of the reaction tube, V/A, is in the range of from 0.1:1 to 100:1, more preferably in the range of from 0.2:1 to 60:1, more preferably in the range of from 0.5:1 to 10:1.

Preferably, (iv) comprises, preferably consists of
(iv.1) supplying the heated mixture obtained from (iii) to the reaction tube;
(iv.2) sealing the reaction tube with the one or two sealing caps;
(iv.3) heating the heated mixture of (iii) and keeping the mixture at the temperature, obtaining a solid material comprising a zeolitic material having framework type CHA and a framework structure comprising Si, Al, O, and H, suspended in its mother liquor, wherein the temperature of the heating medium during heating according to (iv.3) is preferably in the range of from 190 to 235° C., more preferably in the range of from 195 to 230° C., more preferably in the range 200 to 225° C.;
(iv.4) optionally cooling the suspension obtained from (iv.3);
(iv.5) opening one or two sealing caps and removing the suspension obtained from (iv.3) or (iv.4) from the reaction tube.

Preferably, the process of the present invention further comprises
(v) cooling the suspension obtained from (iv), preferably to a temperature in the range of from 15 to 40° C., preferably in the range of from 20 to 30° C.

Preferably, the process of the present invention further comprises
(vi) separating the solid material comprising a zeolitic material having framework type CHA and a framework structure comprising Si, Al, O, and H, obtained from (iv) or (v), preferably from (v), from its mother liquor, obtaining a solid material comprising the zeolitic material having framework type CHA and a framework structure comprising Si, Al, O, and H.

Regarding separating according to (vi), there are no specific restrictions. Preferably, (vi) comprises, more preferably consists of
(vi.1) preferably subjecting the suspension obtained from (iv) or (v), preferably from (v), to solid-liquid separation, obtaining the mother liquor and a solid material comprising the zeolitic material having framework type CHA and a framework structure comprising Si, Al, O, and H; wherein the solid-liquid separation preferably comprises one or more of filtration and centrifugation;
(vi.2) preferably washing the solid material obtained from (vi.1), obtaining a solid material comprising the zeolitic material having framework type CHA and a framework structure comprising Si, Al, O, and H;
(vi.3) drying the solid material obtained from (iv), (v), (vi.1) or (v.2), preferably from (v.2), obtaining a solid material comprising the zeolitic material having framework type CHA and a framework structure comprising Si, Al, O, and H.

Regarding the solid-liquid separation according to (vi.1), it is preferred to subject to membrane filtration or to filtration via a filterpress or via a centrifugal filter. Preferably, wherein (vi) comprises (vi.2), the solid material obtained from (vi.1) is preferably washed with water, preferably with de-ionized water, preferably until the washing water obtained from washing has a pH in the range of from 7 to 8 as determined using a pH sensitive glass electrode. According to (vi.3), drying the solid material preferably comprises preparing a suspension, preferably an aqueous suspension, comprising the solid material obtained from (iv), (v), (vi.1) or (vi.2), preferably from (v), (vi.1) or (vi.2), more preferably from (vi.1) or (vi.2), more preferably from (vi.2), and subjecting the suspension to rapid-drying preferably comprising one or more of spray-drying, spray granulation-drying, and microwave-drying. According to (vi.3), the solid material is preferably dried in a gas atmosphere, preferably having a temperature in the range of from 50 to 150° C., more preferably in the range of from 60 to 120° C., more preferably 70 to 90° C., wherein the gas atmosphere preferably comprises one or more of oxygen and nitrogen, wherein more preferably, the gas atmosphere comprises, more preferably is, one or more of oxygen, air, and lean air.

Preferably, the process of the present invention further comprises
(vii) calcining the solid material obtained from (vi), obtaining a zeolitic material having framework type CHA and a framework structure comprising Si, Al, O, and H.

Preferably, according to (vii), the solid material is calcined in a gas atmosphere, preferably having a temperature in the range of from 500 to 675° C., more preferably in the range of from 550 to 650° C., more preferably 575 to 625° C., wherein the gas atmosphere preferably comprises one or more of oxygen and nitrogen, wherein more preferably, the gas atmosphere comprises, more preferably is, one or more of oxygen, air, and lean air. Preferably, according to (vii), the solid material is heated to a temperature in the range of from 100 to 200° C., more preferably in the range of from 110 to 190° C., more preferably in the range of from 125 to 175° C., kept at a temperature in this range in this range for 0.5 to 6 h, preferably for 0.75 to 4.5 h, more preferably for 1 to 3 h, heated to a temperature in the range of from 500 to 675° C., more preferably in the range of from 550 to 650° C., more preferably 575 to 625° C., and kept at a temperature in this range in this range for 1 to 12 h, preferably for 2.5 to 9 h, more preferably for 3 to 6 h.

Preferably, the process of the present invention further comprises
(viii) cooling the zeolitic material having framework type CHA and a framework structure comprising Si, Al, O, and H, obtained from (vii), preferably to a temperature in the range of from 15 to 40° C., preferably in the range of from 20 to 30° C.

Further, the process of the present invention may comprise
(ix) subjecting the zeolitic material having framework type CHA and a framework structure comprising Si, Al, O, and H as obtained above, to an ion-exchange process, obtaining a mixture comprising a zeolitic material having framework type CHA and comprising M.

According to (ix), one or more ionic non-framework elements contained in the zeolitic material is preferably ion-exchanged, more preferably against one or more cations M, wherein the one or more cations M are cations of one or more of Sr, Zr, Cr, Mg, Mo, Fe, Co, Ni, Cu, Zn, Ru, Rh, Pd, Ag, Os, Ir, and Pt, preferably one or more of Sr, Cr, Mo, Fe, Co, Ni, Cu, Zn, and Ag, more preferably one or more of Cr, Mg, Mo, Fe, Ni, Cu, Zn, and Ag, more preferably one or more of Mg, Mo, Fe, Ni, Cu, Zn, and Ag, more preferably one or more of Cu and Fe, more preferably Cu, and wherein the one or more ionic non-framework elements preferably comprise H and an alkali metal which is preferably one or more of Li, Na, K, and Cs, more preferably one or more of Li, Na, and K, more preferably one or more of Na and K, more preferably Na. Further, (ix) preferably comprises bringing the zeolitic material having framework type CHA and a framework structure comprising Si, Al, O, and H in contact with a solution comprising cations of M, obtaining a mixture comprising the zeolitic material comprising M. Bringing the solution in contact with the zeolitic material according to (ix) may be repeated at least once, for example once, twice, or three times. Preferably, bringing the solution in contact with the zeolitic material according to (ix) comprises one or more of impregnating the zeolitic material with the solution and spraying the solution onto the zeolitic material, preferably impregnating the zeolitic material with the solution.

Further, the process of the present invention may comprise
(x) separating the zeolitic material having framework type CHA and comprising M from the mixture obtained from (ix), wherein (x) preferably comprises
   (x.1) subjecting the mixture obtained from (ix) to a solid-liquid separation method, preferably comprising a filtration method, a centrifugation method, or a spraying method, obtaining the zeolitic material having framework type CHA and comprising M;
   (iii.2) preferably washing the zeolitic material obtained from (iii.1);
   (iii.3) drying the zeolitic material obtained from (iii.1) or (iii.2), preferably from (iii.2).

Further, the process of the present invention may comprise
(xi) calcining the zeolitic material obtained from (x), obtaining the zeolitic material having framework type CHA and comprising M.

Yet further, the process of the present invention may comprise preparing a molding comprising the zeolitic material, wherein said preparing a molding preferably comprises extruding, tabletting, and spraying, wherein more preferably, the molding has a rectangular, a triangular, a hexagonal, a square, an oval or a circular cross section, and/or preferably is in the form of a star, a tablet, a sphere, a cylinder, a strand, or a hollow cylinder.

Further, the present invention relates to a zeolitic material having framework type CHA and a framework structure comprising Si, Al, O, and H, obtainable or obtained by a process as described above.

Yet further, the present invention relates to a zeolitic material having framework type CHA and a framework structure comprising Si, Al, O, and H, preferably the zeolitic material obtainable or obtained by a process as described above, wherein in the framework structure of the zeolitic material, the molar ratio of aluminum relative to silicon, calculated as molar ratio Al₂O₃:SiO₂, is in the range of from 0.001:1 to 0.5:1, preferably in the range of from 0.01:1 to 0.1:1, preferably in the range of from 0.02:1 to 0.05:1, more preferably in the range of from 0.03:1 to 0.04:1.

Preferably, the crystals constituting the zeolitic material have a crystal size, determined via SEM as described in Reference Example 2.4, in the range of from 50 to 1,500 nm, preferably in the range of from 75 to 1,000 nm, more preferably in the range of from 90 to 150 nm, wherein preferably at least 50%m more preferably at least 75%, more preferably at least 90% of the crystals have a size in this range. Preferably, the zeolitic material has a BET specific surface area, determined as described in Reference Example 2.2, of at least 500 m²/g. Preferably, the zeolitic material has a ²⁷Al solid NMR spectrum, determined as described in Reference Example 2.6, exhibiting resonances and a peak maximum in the range of from 62.0 to 54.0 ppm, preferably in the range of from 60.0 to 58.0 ppm, more preferably in the range of from 59.9 to 58.6 ppm, and with a full width at half height of at most 7.0 ppm, preferably at most 5.0 ppm, more preferably at most 4.0 ppm. Preferably, the zeolitic material has a ²⁹Si solid NMR spectrum, determined as described in Reference Example 2.7, exhibiting
resonances and a peak maximum in a first range of from −108.1 to −114.5 ppm, preferably of from −110.3 to −112.2 ppm, more preferably of from −110.9 to −111.7 ppm;
resonances and a peak maximum in a second range of from −102.6 to −108.1 ppm, preferably of from −103.9 to −106.3 ppm, more preferably of from −104.6 to −105.4 ppm;
a resonance with or without a peak maximum in a third range of from −97.7 to −102.6 ppm, preferably of from −99.7 to −101.9 ppm, more preferably of from −100.7 to −101.5 ppm;

wherein the ratio of the integral according to the second range to the integral according to the first range is preferably in the range of from 0.25:1 to 0.45:1, more preferably of from 0.31:1 to 0.39:1, more preferably of from 0.34:1 to 0.36:1.

According to the present invention, the zeolitic material may preferably comprise one or more of Cu and Fe, more preferably Cu.

Generally, the zeolitic material as describe above can be used for every suitable purpose, as an adsorbent, an absorbent, a molecular sieve, a catalytically active material, a catalyst, or a catalyst component, preferably as a catalytically active material, a catalyst, or a catalyst component. Preferred uses include the selective catalytic reduction of nitrogen oxides in an exhaust gas stream, preferably an exhaust gas stream from a diesel engine, the conversion of a Cl compound to one or more olefins, preferably for the conversion of methanol to one or more olefins or the conversion of a synthetic gas comprising carbon monoxide and hydrogen to one or more olefins. Therefore, the present invention also relates to a catalyst, preferably a catalyst for selectively catalytically reducing nitrogen oxides in an exhaust gas stream, preferably an exhaust gas stream from a diesel engine, or for catalytically converting a Cl compound to one or more olefins, preferably converting methanol to one or more olefins or converting a synthetic gas comprising carbon monoxide and hydrogen to one or more olefins, said catalyst comprising the zeolitic material as described above.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”.
1. A batch process for preparing a zeolitic material having framework type CHA and a framework structure comprising Si, Al, O, and H, comprising
   (i) providing a seeding material comprising a zeolitic material having framework type CHA and a framework structure comprising Si, Al, O, and H;
   (ii) preparing a mixture comprising a source of Si, a source of Al, a seeding material provided in (i), a CHA framework structure directing agent comprising a cycloalkylammonium compound, and water,
      wherein the cycloalkylammonium compound is a compound comprising a cation R¹R²R³R⁴N⁺ wherein R¹, R², R³ are, independently from one another, an alkyl residue having from 1 to 6 carbon atoms, and R⁴ is a 5- to 8-membered cycloalkyl residue,
      wherein in mixture, the molar ratio of water relative to Si comprised in the source of Si and in the seeding material, calculated as SiO₂, is in the range of from 5:1 to 15:1,
      wherein the mixture, the molar ratio of sodium, calculated as Na₂O, relative to Si comprised in the source of Si and in the seeding material, calculated as SiO₂, is in the range of from 0:1 to 0.1:1;
   (iii) heating the mixture prepared in (ii) in its liquid state to a temperature of the mixture in the range of from 50 to 90° C. and keeping the liquid mixture at a temperature in this range for 5 to 100 h;
   (iv) heating the heated mixture of (iii) to a temperature of the mixture in the range of from 190 to 230° C. in a crystallization vessel and keeping the mixture at a temperature in this range under autogenous pressure in the crystallization vessel for 0.5 to 10 h, obtaining a solid material comprising a zeolitic material having framework type CHA and a framework structure comprising Si, Al, O, and H, suspended in its mother liquor.
2. The process of embodiment 1, wherein providing the seeding material according to (i) comprises preparing the seeding material by a method comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2).
3. The process of embodiment 1, wherein the seeding material provided according to (i) is obtainable or obtained by a method comprising
   (i.1) providing a zeolitic material having framework type CHA and a framework structure comprising Si, Al, O, and H;
   (i.2) preparing a suspension comprising the zeolitic material provided in (i.1) and a liquid;
   (i.3) milling the suspension prepared in (i.2).
4. The process of embodiment 2 or 3, wherein providing the zeolitic material having framework type CHA and a framework structure comprising Si, Al, O, and H according to (i.1) comprises preparing a zeolitic material having a CHA-type framework structure comprising SiO₂ and Al₂O₃, wherein said process comprises the steps of
   (a) providing a mixture comprising one or more sources for SiO₂, one or more sources for Al₂O₃, one or more tetraalkylammonium cation R¹R²R³R⁴N⁺-containing compounds, and one or more tetraalkylammonium cation R⁵R⁸R⁷R⁸N⁺-containing compounds as structure directing agent;
   (b) crystallizing the obtained mixture, obtaining a zeolitic material having a CHA-type framework structure; wherein R¹, R², R³, R⁴, R⁵, R⁶, and R⁷ independently from one another stand for alkyl, and wherein R⁸ stands for cycloalkyl;
   wherein R¹, R², R³, R⁴, R⁵, R⁶, and R⁷ independently from one another preferably stand for optionally substituted and/or optionally branched (C₁-C₆)alkyl, wherein R⁸ preferably stands for optionally heterocyclic and/or optionally substituted 5- to 8-membered cycloalkyl, wherein the crystallization is preferably conducted under solvothermal conditions,
   wherein the mixture provided in step (1) does not contain any substantial amount of a trimethyl benzyl ammonium containing compound, wherein the mixture provided in step (a) further comprises seed crystals.
5. The process of any one of embodiments 2 to 4, wherein the zeolitic material provided according to (i.1) is preferably the zeolitic material obtainable or obtained by the process of embodiment 4, wherein the zeolitic material preferably exhibits one or more of the following parameters:
   an a first absorption band (B1) in the range of from 3,720 to 3,740 cm⁻¹; and a second absorption band (B2) in the range of from 1,850 to 1,890 cm⁻¹; wherein the ratio of the maximum absorbance of the first absorption band to the second absorption band B1:B2 is comprised in the range of from 0.5 to 1.55;
   the particle size Dv10 of the zeolitic material is in the range of from 400 to 2,500 nm;
   the particle size Dv50 of the zeolitic material is in the range of from 600 to 3,500 nm;
   the particle size Dv90 of the zeolitic material is in the range of from 1,200 to 4,500 nm;
   does not contain any substantial amount of the elements P and/or As;
   the ²⁹Si MAS NMR of the zeolitic material comprises a first peak (P′1) in the range of from −102.0 to −106.0 ppm; and a second peak (P′2) in the range of from −108.0 to −112.5 ppm; wherein the integration of the first and second peaks in the ²⁹Si MAS NMR of the zeolitic material offers a ratio of the integration values P′1:P′2 comprised in the range of from 0.05 to 0.90;
   the SiO₂:Al₂O₃ molar ratio of the framework structure of the zeolitic framework structure is in the range of from 4:1 to 200:1.
6. The process of any one of embodiments 2 to 5, wherein preparing the suspension according to (i.2) comprises admixing the zeolitic material provided in (i.1) with a liquid, wherein the liquid preferably comprises water, wherein more preferably, from 95 to 100 weight-%, more preferably from 99 to 100 weight-% if the liquid consist of water, and wherein the weight ratio of the zeolitic material relative to the liquid is preferably in the range of from 1:10 to 1:50, preferably in the range of from 1:20 to 1:40, more preferably in the range of from 1:25 to 1:35.
7. The process of any one of embodiments 2 to 6, wherein milling the suspension according to (i.3) comprises bead-milling the suspension, preferably for a time in the range of from 10 to 240 min, more preferably in the range of from 20 to 200 min, more preferably in the range of from 30 to 150 min, wherein the beads have a diameter preferably in the range of from 100 to 500 micrometer, more preferably in the range of from 200 to 400 micrometer, more preferably in the range of from 250 to 350 micrometer, and wherein the bead-mill is operated at from 1,000 to 5,000 rpm, preferably from 2,000 to 4,000 rpm, more preferably from 2,500 to 3,500 rpm.
8. The process of any one of embodiments 2 to 7, wherein the method for preparing the seeding material consists of (i.1), (i.2) and (i.3).
9. The process of any one of embodiments 2 to 8, wherein providing the seeding material according to (i) further comprises, after (i.3),
   (i.4) separating the zeolitic material comprised in the milled suspension obtained from (i.3) from the liquid.
10. The process of embodiment 9, wherein separating the zeolitic material from the liquid according to (i.4) comprises subjecting the milled suspension obtained from (i.3) to solid-liquid separation, preferably comprising one or more of filtration and centrifugation, obtaining a separated zeolitic material, and optionally drying the separated zeolitic material in gas atmosphere having a temperature in the range of from 20 to 100° C., wherein the gas atmosphere preferably comprises one or more of oxygen and nitrogen.
11. The process of any one of embodiments 2 to 9, wherein the method for preparing the seeding material consists of (i.1), (i.2), (i.3), and (i.4).
12. The process of any one of embodiments 1 to 11, wherein in the mixture prepared in (ii), the weight ratio of the seeding material, relative to the Si comprised in the source of Si, calculated as SiO₂, is in the range of from 0.025:1 to 0.15:1, preferably in the range of from 0.030:1 to 0.13:1, more preferably in the range of from 0.035:1 to 0.11:1.
13. The process of any one of embodiments 1 to 12, wherein in the mixture prepared in (ii), the weight ratio of the seeding material, relative to the Si comprised in the source of Si, calculated as SiO₂, is in the range of from 0.025:1 to 0.060:1, preferably in the range of from 0.030:1 to 0.055:1, more preferably in the range of from 0.035:1 to 0.045:1.
14. The process of any one of embodiments 1 to 12, wherein in the mixture prepared in (ii), the weight ratio of the seeding material, relative to the Si comprised in the source of Si, calculated as SiO₂, is in the range of from 0.085:1 to 0.15:1, preferably in the range of from 0.090:1 to 0.13:1, more preferably in the range of from 0.095:1 to 0.11:1.
15. The process of any one of embodiments 1 to 14, wherein R¹, R², R³ are, independently from one another, an alkyl residue having from 1 to 5 carbon atoms, preferably from 1 to 4 carbon atoms, more preferably from 1 to 3 carbon atoms, more preferably 1 or 2 carbons, more preferably 1 carbon atom, and wherein R⁴ is a 5- to 7-membered cycloalkyl residue, preferably a 5- or 6-membered cycloalkyl residue, more preferably a 6-membered cycloalkyl residue.
16. The process of any one of embodiments 1 to 15, wherein R¹, R², R³ are methyl and R⁴ is cyclohexyl.
17. The process of any one of embodiments 1 to 16, wherein the cycloalkylammonium compound comprised in the CHA framework structure directing agent according to (ii) comprises, preferably is an ammonium salt, preferably one or more of a halide, a sulfate, a nitrate, an acetate, and a hydroxide, more preferably one or more of a chloride, a bromide, a sulfate, a nitrate, an acetate, and a hydroxide, more preferably one or more of a chloride, a bromide, and a hydroxide, more preferably a hydroxide.
18. The process of any one of embodiments 1 to 17, wherein the cycloalkylammonium compound comprised in the CHA framework structure directing agent according to (ii) comprises, preferably is N,N,N-trimethyl-cyclohexylammonium hydroxide.
19. The process of any one of embodiments 1 to 18, wherein from 99 to 100 mol-%, preferably from 99.5 to 100 mol-%, more preferably from 99.9 to 100 mol-% of the CHA framework structure directing agent consist of N,N,N-trimethyl-cyclohexylammonium hydroxide, wherein more preferably, the CHA framework structure directing agent is free of N,N,N-trimethyl-1-adamantylammonium hydroxide, preferably free of an N,N,N-trimethyl-1-adamantylammonium comprising compound, more preferably free of an adamantylammonium comprising compound.
20. The process of any one of embodiments 1 to 19, wherein the CHA framework structure directing agent according to (ii) further comprises a tetraalkylammonium compound comprising a cation R⁵R⁶R⁷R⁸N⁺ wherein R⁵, R⁶, R⁷, R⁸ are, independently from one another, an optionally substituted alkyl residue having from 1 to 4 carbon atoms, preferably from 1 to 3 carbon atoms, more preferably 1 or 2 carbons, wherein more preferably, R⁵, R⁶, R⁷, R⁸ are methyl, wherein the substituent is preferably one or more of chloro and hydroxyl, more preferably hydroxyl.
21. The process of embodiment 20, wherein the tetraalkylammonium compound comprised in the CHA framework structure directing agent according to (ii) comprises, preferably is an ammonium salt, preferably one or more of a halide, a sulfate, a nitrate, an acetate, and a hydroxide, more preferably one or more of a chloride, a bromide, a sulfate, a nitrate, an acetate, and a hydroxide, more preferably one or more of a chloride, a bromide, and a hydroxide, more preferably a hydroxide.
22. The process of embodiment 20 or 21, wherein the tetraalkylammonium compound comprised in the CHA framework structure directing agent according to (ii) comprises, preferably is tetramethylammonium hydroxide.
23. The process of any one of embodiments 20 to 22, wherein in the CHA framework structure directing agent according to (ii), the molar ratio of the cycloalkylammonium compound relative to the tetraalkylammonium compound is in the range of from 1:1 to 5.5:1, preferably in the range of from 1.1:1 to 4:1, more preferably in the range of from 1.3:1 to 3:1, more preferably in the range of from 1.5:1 to 2.0:1.
24. The process of any one of embodiments 20 to 23, wherein from 99 to 100 mol-%, preferably from 99.5 to 100 mol-%, more preferably from 99.9 to 100 mol-% of the CHA framework structure directing agent consist of the cycloalkylammonium compound and the tetraalkylammonium compound, wherein more preferably, the CHA framework structure directing agent is free of N,N,N-trimethyl-1-adamantylammonium hydroxide, preferably free of an N,N,N-trimethyl-1-adamantylammonium comprising compound, more preferably free of an adamantylammonium comprising compound.
25. The process of any one of embodiments 1 to 24, wherein in the mixture prepared in (ii), the molar ratio of the CHA framework structure directing agent relative to Si comprised in the source of Si and in the seeding material, calculated as SiO₂, is in the range of from 0.20:1 to 0.30:1, preferably in the range of from 0.22:1 to 0.29:1, more preferably in the range of from 0.25:1 to 0.28:1.
26. The process of any one of embodiments 1 to 25, wherein in mixture prepared in (ii), the molar ratio of water relative to Si comprised in the source of Si and in the seeding material, calculated as SiO₂, is in the range of from 7:1 to 15:1, preferably in the range of from 9:1 to 12:1.
27. The process of any one of embodiments 1 to 26, wherein the source of Si comprises, preferably is, one or more of a silica and a silicate, preferably one or more of a fumed silica, a silica sol, an amorphous silica, a silica gel, a silicic acid, a silic acid ester, colloidal silica, a tetraalkoxysilane, a disilicate, and a sesquisilcate, more preferably one or more of a fumed silica, a silica hydrosol, a silica gel, a silicic acid, a silicic acid ester, a colloidal silica, and a tetraalkoxysilane, more preferably one or more of a fumed silica, a silica hydrosol, a silica gel, and a colloidal silica, more preferably one or more of a fumed silica, a silica gel, and a colloidal silica.
28. The process of any one of embodiments 1 to 27, wherein the source of Si comprises, more preferably is, a colloidal silica.
29. The process of any one of embodiments 1 to 28, wherein the source of Al comprises, preferably is, one or more of an alumina, an aluminate, and an aluminum salt, preferably one or more of an alumina and an aluminum salt, more preferably one or more of an alumina, a tri(C1-C5)alkoxide, an AlO(OH), an Al(OH)₃, an aluminum halide wherein the aluminum halide is preferably one or more of an aluminum fluoride, an aluminum chloride and an aluminum bromide, an aluminum sulfate, an aluminum phosphate, and an aluminum fluorosilicate, more preferably one or more of an AlO(OH) and an Al(OH)₃.
30. The process of any one of embodiments 1 to 29, wherein the source of Al comprises, more preferably is, an Al(OH)₃, preferably a crystalline Al(OH)₃, more preferably gibbsite.
31. The process of any one of embodiments 1 to 30, wherein in the mixture prepared in (ii), the molar ratio of the source of Al, calculated as Al₂O₃, relative to Si comprised in the source of Si and in the seeding material, calculated as SiO₂, is in the range of from 0.001:1 to 0.5:1, preferably in the range of from 0.01:1 to 0.1:1, preferably in the range of from 0.02:1 to 0.05:1, more preferably in the range of from 0.03:1 to 0.04:1.
32. The process of any one of embodiments 1 to 31, wherein from 95 to 100 weight-%, preferably from 98 to 100 weight-%, more preferably from 99 to 100 weight-% of the mixture prepared in (ii) consist of the source of Si, the source of Al, the seeding material provided in (i), the CHA framework structure directing agent comprising a cycloalkylammonium compound, and the water.
33. The process of any one of embodiments 1 to 32, wherein from 99.5 to 100 weight-%, preferably from 99.9 to 100 weight-%, more preferably from 99.9 to 100 weight-% of the mixture prepared in (ii) consist of the source of Si, the source of Al, the seeding material provided in (i), the CHA framework structure directing agent comprising a cycloalkylammonium compound, and the water.
34. The process of any one of embodiments 1 to 33, wherein the mixture prepared in (ii), the molar ratio of phosphorus, calculated as elemental P, relative to Si comprised in the source of Si and in the seeding material, calculated as SiO₂, is in the range of from 0:1 to 0.001:1.
35. The process of any one of embodiments 1 to 34, wherein preparing the mixture according to (ii) comprises
   (ii.1) preparing a mixture comprising the source of Si, the source of Al, the CHA framework structure directing agent comprising a cycloalkylammonium compound, and water;
   (ii.2) adding the seeding material to the mixture prepared in (ii.1), obtaining the mixture to be subjected to (iii).
36. The process of embodiment 35, wherein (ii.1) comprises
   (ii.1.1) preparing a mixture comprising the source of Al and the CHA framework structure directing agent comprising a cycloalkylammonium compound;
   (ii.1.2) agitating the mixture prepared in (ii.1.1) at a temperature of the mixture in the range of from 10 to 50° C. for 5 to 60 min;
   (ii.1.3) adding the source of Si to the mixture obtained from (ii.1.2);
   (ii.1.4) agitating the mixture prepared in (ii.1.3) at a temperature of the mixture in the range of from 10 to 50° C. for 1 to 30 min, obtaining the mixture to be subjected to (iii).
37. The process of embodiment 36, wherein according to (ii.1.2), the mixture is agitated at a temperature of the mixture in the range of from 15 to 40° C., preferably in the range of from 20 to 30° C.
38. The process of embodiment 36 or 37, wherein according to (ii.1.2), the mixture is agitated for 10 to 50 min, preferably for 20 to 40 min.
39. The process of any one of embodiments 36 to 38, wherein according to (ii.1.2), the mixture is agitated at a pressure in the range of from 0.7 to 2 bar(abs), preferably in the range of from 0.8 to 1.5 bar(abs), more preferably in the range of from 0.9 to 1.1 bar(abs).
40. The process of any one of embodiments 36 to 39, wherein agitating according to (ii.1.2) comprises mechanically agitating the mixture, preferably stirring the mixture.
41. The process of embodiment 40, wherein according to (ii.1.2), the mixture is stirred at from 100 to 1,000 rpm, preferably from 200 to 750 rpm, more preferably from 400 to 600 rpm.
42. The process of any one of embodiments 36 to 41, wherein according to (ii.1.4), the mixture is agitated at a temperature of the mixture in the range of from 15 to 40° C., preferably in the range of from 20 to 30° C.
43. The process of any one of embodiment 36 to 42, wherein according to (ii.1.4), the mixture is agitated for 2 to 20 min, preferably for 5 to 15 min.
44. The process of any one of embodiments 36 to 43, wherein according to (ii.1.4), the mixture is agitated at a pressure in the range of from 0.7 to 2 bar(abs), preferably in the range of from 0.8 to 1.5 bar(abs), more preferably in the range of from 0.9 to 1.1 bar(abs).
45. The process of any one of embodiments 36 to 44, wherein agitating according to (ii.1.4) comprises mechanically agitating the mixture, preferably stirring the mixture.
46. The process of embodiment 45, wherein according to (ii.1.4), the mixture is stirred at from 100 to 1,000 rpm, preferably from 200 to 750 rpm, more preferably from 400 to 600 rpm.
47. The process of any one of embodiments 36 to 46, wherein (ii.1) consists of (ii.1.1), (ii.1.2), (ii.1.3) and (ii.1.4).
48. The process of any one of embodiments 35 to 47, wherein (ii) consists of (ii.1) and (ii.2).
49. The process of any one of embodiments 1 to 48, wherein according to (iii), the mixture prepared (ii) is heated in its liquid state to a temperature of the mixture in the range of from 55 to 80° C., preferably in the range of from 60 to 70° C.
50. The process of any one of embodiments 1 to 49, wherein according to (iii), the liquid mixture is kept at the temperature for 5 to 80 h, preferably for 20 to 50 h.
51. The process of any one of embodiments 1 to 50, wherein heating the mixture according to (iii), preferably heating the mixture and keeping the mixture at the temperature according to (iii) is carried out at a pressure in the range of from 0.7 to 2 bar(abs), preferably in the range of from 0.8 to 1.5 bar(abs), more preferably in the range of from 0.9 to 1.1 bar(abs).
52. The process of any one of embodiments 1 to 51, wherein according to (iii), the mixture is heated to the temperature at a temperature ramp in the range of from 0.2 to 5 K/min, preferably in the range of from 0.5 to 4 K/min more preferably in the range of from 1 to 3 K/min.
53. The process of any one of embodiments 1 to 52, wherein during keeping the mixture at the temperature according to (iii), preferably during heating the mixture and keeping the mixture at the temperature according to (iii), the mixture is agitated.
54. The process of embodiment 53, wherein agitating according to (iii) comprises mechanically agitating the mixture, preferably stirring the mixture.
55. The process of embodiment 54, wherein according to (iii), the mixture is stirred at from 100 to 1,000 rpm, preferably from 200 to 750 rpm, more preferably from 400 to 600 rpm.
56. The process of any one of embodiments 1 to 55, wherein (iii) consists of heating the mixture and keeping the mixture at the temperature.
57. The process of any one of embodiments 1 to 56, wherein according to (iv), the mixture of (iii) is heated to a temperature of the mixture in the range of from 195 to 225° C., preferably in the range 200 to 220° C.
58. The process of any one of embodiments 1 to 57, wherein according to (iv), the mixture is kept at the temperature for 0.75 to 7.5 h, preferably for 1 to 5 h.
59. The process of any one embodiments 1 to 58, wherein according to (iv), the mixture is heated to the temperature at a temperature ramp in the range of from 0.1 to 20 K/min, preferably in the range of from 0.5 to 15 K/min, preferably in the range of from 1 to 10 K/min.
60. The process of any one of embodiments 1 to 59, wherein during heating the mixture or keeping the mixture at the temperature according to (iv), preferably during heating the mixture and keeping the mixture at the temperature according to (iv), the mixture as agitated, preferably mechanically agitated, wherein more preferably, the crystallization vessel is agitated.
61. The process of any one of embodiments 1 to 60, wherein the crystallization vessel according to (iv) is an autoclave.
62. The process of any one of embodiments 1 to 60, wherein the crystallization vessel according to (iv) is a tubular reactor comprising a reaction tube and one or two sealing caps for sealing the reaction tube.
63. The process of embodiment 62, wherein during heating the mixture and keeping the mixture at the temperature according to (iv), the tubular reactor is externally heated by a heating medium.
64. The process of embodiment 63, wherein the heating medium is a gaseous heating medium, a liquid heating medium, or a solid heating medium, preferably a gaseous heating medium or a liquid heating medium.
65. The process of embodiment 64, wherein the heating medium is a gaseous heating medium, preferably contained in a statically or a continuously operated oven, wherein during heating and keeping at the temperature according to (iv), the gaseous heating medium is statically or continuously brought in contact with the reaction tube.
66. The process of embodiment 64, wherein the heating medium is a liquid heating medium, preferably comprising an oil, said heating medium more preferably being contained in a statically or a continuously operated bath, wherein during heating and keeping at the temperature according to (iv), the liquid heating medium is statically or continuously brought in contact with the reaction tube.
67. The process of any one of embodiments 62 to 66, wherein the reaction tube is made of material having a thermal diffusivity in the range of from 3×10⁻⁶ to 30×10⁻⁶ m²/s, preferably in the range of from 5×10⁻⁶ to 25×10⁻⁶ m²/s.
68. The process of embodiment 67, wherein the material is stainless steel.
69. The process of any one of embodiments 62 to 68, wherein the ratio of the volume V/cm³ of the reaction tube to the outer surface area A/cm² of the reaction tube, V/A, is in the range of from 0.1:1 to 100:1, preferably in the range of from 0.2:1 to 60:1, more preferably in the range of from 0.5:1 to 10:1.
70. The process of any one of embodiments 62 to 69, wherein (iv) comprises, preferably consists of
   (iv.1) supplying the heated mixture obtained from (iii) to the reaction tube;
   (iv.2) sealing the reaction tube with the one or two sealing caps;
   (iv.3) heating the heated mixture of (iii) and keeping the mixture at the temperature, obtaining a solid material comprising a zeolitic material having framework type CHA and a framework structure comprising Si, Al, O, and H, suspended in its mother liquor;
   (iv.4) optionally cooling the suspension obtained from (iv.3);
   (iv.5) opening one or two sealing caps and removing the suspension obtained from (iv.3) or (iv.4) from the reaction tube.
71. The process of embodiment 70, wherein the temperature of the heating medium during heating according to (iv.3) is in the range of from 190 to 235° C., preferably in the range of from 195 to 230° C., more preferably in the range 200 to 225° C.
72. The process of any one of embodiments 1 to 71, further comprising
   (v) cooling the suspension obtained from (iv), preferably to a temperature in the range of from 15 to 40° C., preferably in the range of from 20 to 30° C.
73. The process of any one of embodiments 1 to 72, further comprising
   (vi) separating the solid material comprising a zeolitic material having framework type CHA and a framework structure comprising Si, Al, O, and H, obtained from (iv) or (v), preferably from (v), from its mother liquor, obtaining a solid material comprising the zeolitic material having framework type CHA and a framework structure comprising Si, Al, O, and H.
74. The process of embodiment 73, wherein (vi) comprises, preferably consists of
   (vi.1) preferably subjecting the suspension obtained from (iv) or (v), preferably from (v), to solid-liquid separation, obtaining the mother liquor and a solid material comprising the zeolitic material having framework type CHA and a framework structure comprising Si, Al, O, and H;
   (vi.2) preferably washing the solid material obtained from (vi.1), obtaining a solid material comprising the zeolitic material having framework type CHA and a framework structure comprising Si, Al, O, and H;
   (vi.3) drying the solid material obtained from (iv), (v), (vi.1) or (v.2), preferably from (v.2), obtaining a solid material comprising the zeolitic material having framework type CHA and a framework structure comprising Si, Al, O, and H.
75. The process of embodiment 74, wherein (vi) comprises (vi.1), wherein solid-liquid separation according to (vi.1) comprises one or more of filtration and centrifugation.
76. The process of embodiment 74 or 75, wherein (vi) comprises (vi.2), wherein the solid material obtained from (vi.1) is washed with water, preferably with de-ionized water, preferably until the washing water obtained from washing has a pH in the range of from 7 to 8 as determined using a pH sensitive glass electrode.
77. The process of any one of embodiments 74 to 76, wherein according to (vi.3), drying the solid material comprises preparing a suspension, preferably an aqueous suspension, comprising the solid material obtained from (iv), (v), (vi.1) or (vi.2), preferably from (v), (vi.1) or (vi.2), more preferably from (vi.1) or (vi.2), more preferably from (vi.2), and subjecting the suspension to rapid-drying preferably comprising one or more of spray-drying, spray granulation-drying, and microwave-drying.
78. The process of any one of embodiments 74 to 77, wherein according to (vi.3), the solid material is dried in a gas atmosphere, preferably having a temperature in the range of from 50 to 150° C., more preferably in the range of from 60 to 120° C., more preferably 70 to 90° C., wherein the gas atmosphere preferably comprises one or more of oxygen and nitrogen, wherein more preferably, the gas atmosphere comprises, more preferably is, one or more of oxygen, air, and lean air.
79. The process of any one of embodiments 73 to 78, further comprising
   (vii) calcining the solid material obtained from (vi), obtaining a zeolitic material having framework type CHA and a framework structure comprising Si, Al, O, and H.
80. The process of embodiment 79, wherein according to (vii), the solid material is calcined in a gas atmosphere, preferably having a temperature in the range of from 500 to 675° C., more preferably in the range of from 550 to 650° C., more preferably 575 to 625° C., wherein the gas atmosphere preferably comprises one or more of oxygen and nitrogen, wherein more preferably, the gas atmosphere comprises, more preferably is, one or more of oxygen, air, and lean air.
81. The process of embodiment 80 wherein according to (vii), the solid material is heated to a temperature in the range of from 100 to 200° C., preferably in the range of from 110 to 190° C., more preferably in the range of from 125 to 175° C., kept at a temperature in this range in this range for 0.5 to 6 h, preferably for 0.75 to 4.5 h, more preferably for 1 to 3 h, heated to a temperature in the range of from 500 to 675° C., more preferably in the range of from 550 to 650° C., more preferably 575 to 625° C., and kept at a temperature in this range in this range for 1 to 12 h, preferably for 2.5 to 9 h, more preferably for 3 to 6 h.
82. The process of any one of embodiments 79 to 81, further comprising
   (viii) cooling the zeolitic material having framework type CHA and a framework structure comprising Si, Al, O, and H, obtained from (vii), preferably to a temperature in the range of from 15 to 40° C., preferably in the range of from 20 to 30° C.
83. The process of embodiment 82, further comprising
   (ix) subjecting the zeolitic material having framework type CHA and a framework structure comprising Si, Al, O, and H of any one of embodiments 79 to 82, preferably of embodiment 82, to an ion-exchange process, obtaining a mixture comprising a zeolitic material having framework type CHA and comprising M.
84. The process of embodiment 83, wherein according to (ix), one or more ionic non-framework elements contained in the zeolitic material is ion-exchanged, preferably against one or more cations M, wherein the one or more cations M are cations of one or more of Sr, Zr, Cr, Mg, Mo, Fe, Co, Ni, Cu, Zn, Ru, Rh, Pd, Ag, Os, Ir, and Pt, preferably one or more of Sr, Cr, Mo, Fe, Co, Ni, Cu, Zn, and Ag, more preferably one or more of Cr, Mg, Mo, Fe, Ni, Cu, Zn, and Ag, more preferably one or more of Mg, Mo, Fe, Ni, Cu, Zn, and Ag, more preferably one or more of Cu and Fe, more preferably Cu, and wherein the one or more ionic non-framework elements preferably comprise H and an alkali metal which is preferably one or more of Li, Na, K, and Cs, more preferably one or more of Li, Na, and K, more preferably one or more of Na and K, more preferably Na.
85. The process of embodiment 83 or 84, wherein (ix) comprises bringing the zeolitic material having framework type CHA and a framework structure comprising Si, Al, O, and H in contact with a solution comprising cations of M, obtaining a mixture comprising the zeolitic material comprising M;
86. The process of embodiment 85, wherein bringing the solution in contact with the zeolitic material according to (ix) is repeated at least once.
87. The process of embodiment 85 or 86, wherein bringing the solution in contact with the zeolitic material according to (ix) comprises one or more of impregnating the zeolitic material with the solution and spraying the solution onto the zeolitic material, preferably impregnating the zeolitic material with the solution.
88. The process of any one of embodiments 83 to 87, further comprising (x) separating the zeolitic material having framework type CHA and comprising M from the mixture obtained from (ix).
89. The process of embodiment 88, wherein separating the zeolitic material according to (x) comprises
   (x.1) subjecting the mixture obtained from (ix) to a solid-liquid separation method, preferably comprising a filtration method, a centrifugation method, or a spraying method, obtaining the zeolitic material having framework type CHA and comprising M;
   (iii.2) preferably washing the zeolitic material obtained from (iii.1);
   (iii.3) drying the zeolitic material obtained from (iii.1) or (iii.2), preferably from (iii.2).
90. The process of embodiment 89, further comprising
   (xi) calcining the zeolitic material obtained from (x), obtaining the zeolitic material having framework type CHA and comprising M.
91. The process of any one of embodiments 1 to 90, further comprising preparing a molding comprising the zeolitic material, said preparing a molding preferably comprising extruding, tabletting, and spraying, wherein more preferably, the molding has a rectangular, a triangular, a hexagonal, a square, an oval or a circular cross section, and/or preferably is in the form of a star, a tablet, a sphere, a cylinder, a strand, or a hollow cylinder.
92. A zeolitic material having framework type CHA and a framework structure comprising Si, Al, O, and H, obtainable or obtained by a process according to any one of embodiments 1 to 90.
93. A zeolitic material having framework type CHA and a framework structure comprising Si, Al, O, and H, preferably the zeolitic material of embodiment 92, wherein in the framework structure of the zeolitic material, the molar ratio of aluminum relative to silicon, calculated as molar ratio Al₂O₃:SiO₂, is in the range of from 0.001:1 to 0.5:1, preferably in the range of from 0.01:1 to 0.1:1, more preferably in the range of from 0.02:1 to 0.05:1, more preferably in the range of from 0.03:1 to 0.04:1.
94. The zeolitic material of embodiment 92 or 93, obtainable or obtained by a process according to any one of embodiments 1 to 82, wherein the process preferably consists of (i), (ii), (iii), (iv), (v), (vi), (vii) and preferably (viii).
95. The zeolitic material of any one of embodiment 92 to 94, wherein the crystals constituting the zeolitic material have a crystal size, determined via SEM as described in Reference Example 2.4, in the range of from 50 to 1,500 nm, preferably in the range of from 75 to 1,000 nm, more preferably in the range of from 90 to 150 nm, wherein preferably at least 50%m more preferably at least 75%, more preferably at least 90% of the crystals have a size in this range.
96. The zeolitic material of any one of embodiment 92 to 95, having a BET specific surface area, determined as described in Reference Example 2.2, of at least 500 m²/g.
97. The zeolitic material of any one of embodiments 92 to 96, having a ²⁷Al solid NMR spectrum, determined as described in Reference Example 2.6, exhibiting resonances and a peak maximum in the range of from 62.0 to 54.0 ppm, preferably in the range of from 60.0 to 58.0 ppm, more preferably in the range of from 59.9 to 58.6 ppm, and with a full width at half height of at most 7.0 ppm, preferably at most 5.0 ppm, more preferably at most 4.0 ppm.
98. The zeolitic material of any one of embodiments 92 to 97, having a ²⁹Si solid NMR spectrum, determined as described in Reference Example 2.7, exhibiting
   resonances and a peak maximum in a first range of from −108.1 to −114.5 ppm, preferably of from −110.3 to −112.2 ppm, more preferably of from −110.9 to −111.7 ppm;
   resonances and a peak maximum in a second range of from −102.6 to −108.1 ppm, preferably of from −103.9 to −106.3 ppm, more preferably of from −104.6 to −105.4 ppm;
   a resonance with or without a peak maximum in a third range of from −97.7 to −102.6 ppm, preferably of from −99.7 to −101.9 ppm, more preferably of from −100.7 to −101.5 ppm;
   wherein the ratio of the integral according to the second range to the integral according to the first range is preferably in the range of from 0.25:1 to 0.45:1, more preferably of from 0.31:1 to 0.39:1, more preferably of from 0.34:1 to 0.36:1.
99. The zeolitic material of embodiment 92 or 93, obtainable or obtained by a process according to any one of embodiments 83 to 90, wherein the process preferably consists of (i), (ii), (iii), (iv), (v), (vi), (vii), preferably (viii), (ix), and preferably (x).
100. The zeolitic material of embodiment 99, comprising one or more of Cu and Fe, preferably Cu.
101. Use of a zeolitic material according to any one of embodiments 92 to 100 as an adsorbent, an absorbent, a molecular sieve, a catalytically active material, a catalyst, or a catalyst component, preferably as a catalytically active material, a catalyst, or a catalyst component.
102. The use of embodiment 101 for the selective catalytic reduction of nitrogen oxides in an exhaust gas stream, preferably an exhaust gas stream from a diesel engine.
103. The use of embodiment 101 for the conversion of a Cl compound to one or more olefins, preferably for the conversion of methanol to one or more olefins or the conversion of a synthetic gas comprising carbon monoxide and hydrogen to one or more olefins.
104. A method for selectively catalytically reducing nitrogen oxides in an exhaust gas stream, preferably an exhaust gas stream from a diesel engine, said method comprising bringing said exhaust gas stream in contact with a catalyst comprising the zeolitic material according to any one of embodiments 92 to 100, preferably according to embodiment 99 and 100.
105. A method for selectively catalytically reducing nitrogen oxides in an exhaust gas stream, preferably an exhaust gas stream from a diesel engine, said method comprising preparing a zeolitic material by a process according to any one of embodiments 1 to 90, preferably according to any one of embodiments 83 to 90, and bringing said exhaust gas stream in contact with a catalyst comprising said zeolitic material.
106. A method for catalytically converting a Cl compound to one or more olefins, preferably converting methanol to one or more olefins or converting a synthetic gas comprising carbon monoxide and hydrogen to one or more olefins, said method comprising bringing said Cl compound in contact with a catalyst comprising the zeolitic material according to any one of embodiments 92 to 100.
107. A method for catalytically converting a Cl compound to one or more olefins, preferably converting methanol to one or more olefins or converting a synthetic gas comprising carbon monoxide and hydrogen to one or more olefins, said method comprising preparing a zeolitic material by a process according to any one of embodiments 1 to 90, and bringing said Cl compound in contact with a catalyst comprising said zeolitic material.
108. A catalyst, preferably a catalyst for selectively catalytically reducing nitrogen oxides in an exhaust gas stream, preferably an exhaust gas stream from a diesel engine, or for catalytically converting a Cl compound to one or more olefins, preferably converting methanol to one or more olefins or converting a synthetic gas comprising carbon monoxide and hydrogen to one or more olefins, said catalyst comprising the zeolitic material according to any one of embodiments 92 to 100.

The present invention is further illustrated by the following reference examples, examples, and comparative examples.

### EXAMPLES

### Reference Example 1: Preparing a Zeolitic Seed Material Having Framework Type CHA

A zeolitic seed material was prepared according to a conventional synthesis procedure as described in WO 2015/185625 A1, Example 3, on page 51.

As seed material used for this synthesis, a zeolitic material used prepared according to the following method: 2,040 kg of water were placed in a stirring vessel and 3,924 kg of a solution of 1-adamantyltrimethylammonium hydroxide (20 weight-% aqueous solution) were added thereto under stirring. 415.6 kg of a solution of sodium hydroxide (20 weight-% aqueous solution) were then added, followed by 679 kg of aluminum triisopropylate (Dorox® D 10, from Ineos), after which the resulting mixture was stirred for 5 min. 7,800.5 kg of a solution of colloidal silica (40 weight-% aqueous solution; Ludox® AS40, Sigma Aldrich) were then added and the resulting mixture stirred for 15 min before being transferred to an autoclave. 1,000 kg of distilled water used for washing out the stirring vessel were added to the mixture in the autoclave, and the final mixture was then heated under stirring for 16 h at 170° C. The solid product was then filtered off and the filter cake washed with distilled water. The resulting filter cake was then dispersed in distilled water in a spray dryer mix tank to obtain a slurry with a solids concentration of approximately 24% and the spray-dried, wherein the inlet temperature was set to 477-482° C. and the outlet temperature was measured to be 127-129° C., thus affording a spray-dried powder of a zeolite having the CHA framework structure.

The resulting material had a BET specific surface area of 558 m²/g, and a crystallinity of 105% as determined by powder X-ray diffraction. The sodium content of the product was determined to be 0.75 weight-% calculated as Na₂O. The zeolitic material had a molar silica:alumina ratio (SiO₂:Al₂O₃) of 34:1.

### Reference Example 2.1: Determination of the Particle Distribution Dv

The volume-based particle size distribution Dv of the samples was performed by dispersing 0.1 g of the zeolite powder in 100 g H₂O and treating by ultrasound for 10 minutes. The dynamic light scattering was performed on a Zetasizer Nano ZS with the Malvern Zeta Sizer Software, Version 6.34, applying 5 runs a 10 second measurement time for each sample. The given values are the average particle size by number in nanometer.

### Reference Example 2.2: Determination of the BET Specific Surface Area

The BET specific surface area of the alumina was determined according to DIN 66131 or DIN-ISO 9277 using liquid nitrogen.

### Reference Example 2.3: Determination of the XRD Patterns and the Crystallinity

Powder X-ray diffraction (XRD) patterns were conducted using a diffractometer (Rigaku Ultima IV) equipped with a D/Tex Ultra detector operated with Cu Kα monochromatized radiation at 40 kV and 40 mA. A scan step was 0.02° at a scan speed of 20°/min. Crystallinity was calculated using integrated peak areas of the peaks in 2theta rage of 20°-35°.

### Reference Example 2.4: Determination of the SEM Images

Field-emission scanning electron microscope (FE-SEM) images were observed on a JSM-7500FA (JEOL) after Os coating over the powder samples on the carbon tape.

### Reference Example 2.5: Determination of the ICP (Elemental Analysis)

Elemental analysis (Si/AI ratio) was performed on a Thermo Scientific iCAP-6300 inductively coupled plasma-atomic emission spectrometer (ICP-AES) after dissolving the products in a potassium hydroxide solution. Typically, about 10 mg of the samples were dissolved in 1 ml of 50% potassium hydroxide solution and then diluted with deionized water to the final volume of 100 mL.

### Reference Example 2.6: Determination of the ²⁷Al MAS Solid-State NMR

²⁷Al solid-state NMR spectra were recorded at 9.4 Tesla under 10 kHz magic-angle spinning using a 15°-single-pulse-acquisition sequence with 0.5 s repetition time for 2 h. The sample was stored at 62% relative humidity for at least 60 h prior to measurement. Resonances were indirectly referenced to Al(NO₃)₃ in D₂O, 1.1 mol/kg, as zero reference, with a frequency of 0.26056859 on the unified shift scale, in line with IU PAC recommendations 2008 (Pure Appl. Chem., Vol. 80, No. 1, pp. 59-84, 2008), using external secondary standards.

### Reference Example 2.7: Determination of the ²⁹Si MAS Solid-State NMR

²⁹Si solid-state NMR spectra were recorded at 7 Tesla under 5 kHz magic-angle spinning using a 90°-single-pulse-acquisition sequence with heteronuclear radio-frequency proton-decoupling during acquisition and 120 s repetition time for 16 h. The sample was stored at 62% relative humidity for at least 60 h prior to measurement. Resonances were indirectly referenced to Me₄Si in CDCl₃, volume fraction 1%, as zero reference, with a frequency of 0.19867187 on the unified shift scale, in line with IU PAC recommendations 2008 (Pure Appl. Chem., Vol. 80, No. 1, pp. 59-84, 2008), using external secondary standards.

### Reference Example 2.8: IR Spectra

The IR-spectra were obtained from samples free of a carrier material, wherein said sample were heated at 300° C. in high vacuum for 3 h prior to measurement. The measurements were performed using a Nicolet 6700 spectrometer in a high vacuum measurement cell with CaF₂ windows. The obtained data was transformed to absorbance values, and the analysis was performed on the spectra after base line correction.

### Comparative Example 1: Conventional Process Without Aging in an Autoclave, Using Aluminum Isopropoxide as Source of Al

1.991 g of aqueous N,N,N-trimethylcyclohexylammonium hydroxide (CHTMAOH) solution (20 weight-%) and 0.655 g of aqueous tetramethylammonium hydroxide solution (TMAOH) (25 weight-%) were first mixed. Then, 0.295 g of aluminum isopropoxide was added slowly under stirring at room temperature for 30 min at 500 rpm. After dissolution of the aluminum isopropoxide, 3.020 g of colloidal silica (Ludox® AS-40) were added. The mixture was further stirred for 10 min at room temperature at 500 rpm before the addition of 0.046 g of CHA seed crystals prepared according to Reference Example 1. The mixture was charged to a 23-mL Teflon-lined autoclave. The tightly closed autoclave was placed in an oven pre-heated at 175° C. Hydrothermal treatment was carried out at 175° C. with 20 rpm tumbling for 20 h, 24 h, and 48 h. Samples were collected using centrifugation at 14,000 rpm and washed with water until the pH of the washing water was in the range of from 7-8. The solid products was dried in air at 80° C. and calcined in air at 600° C. for 5 h.

The SEM of the material obtained after a synthesis time of 48 h is shown in FIG. 5. The XRD of the obtained materials after 20 h, 24 h and 48 h is shown in FIG. 6. The crystallinity, determined as described in Reference Example 2.3, was only 17.2% after a crystallization time of 20 h (bottom curve) and only 27.3% after a crystallization time 24 h (middle curve). A reasonable crystallinity of about 100% was obtained only after a crystallization time of 48 h (top curve).

### Comparative Example 2: Conventional Process Without Aging in an Autoclave, Using Aluminum Isopropoxide as Source of Al

1.998 g of aqueous CHTMAOH solution (20 weight-%) and 0.656 g of aqueous TMAOH solution (25 weight-%) were first mixed. Then, 0.295 g of aluminum isopropoxide were added slowly under stirring. After dissolution of aluminum isopropoxide under stirring for 30 min at 500 rpm, 3.008 g of Ludox® AS-40 were added. The mixture was further stirred for 10 min at room temperature at 500 rpm before the addition of 0.046 g of CHA seed crystals prepared according to Reference Example 1. The mixture was charged to a 23-ml Teflon-lined autoclave. The tightly closed autoclave was placed in an oven pre-heated at 200° C. Hydrothermal treatment was carried out at 200° C. at 60 rpm tumbling for 3 h, 5 h, 16 h, and 20 h. Samples were collected using centrifugation at 14,000 rpm and washed with water until the pH of the washing water was in the range of from 7-8. The solid products were dried at 80° C. and calcined in air at 600° C. for 5 h.

The SEM of the material obtained after a synthesis time of 48 h is shown in FIG. 7. The XRD of the obtained materials after 3 h, 5 h, 16 h and 20 h is shown in FIG. 8. The crystallinity, determined as described in Reference Example 2.3, was only 1.7% after a crystallization time of 3 h and 11.7% after a crystallization time of 5 h (bottom curves). A reasonable crystallinity of about 100% was obtained only after a crystallization time of 16 h and 20 h (middle curve and top curve).

### Comparative Example 3: Conventional Process Without Aging in an Autoclave, Using Aluminum Hydroxyide as Source of Al

1.997 g of an aqueous CHTMAOH solution (20 weight-%) and 0.656 g of aqueous TMAOH solution (25 weight-%) were first mixed. Then, 0.112 g of aluminum hydroxide (gibbsite) were added slowly under stirring. After stirring the mixture for 30 min at room temperature at 500 rpm, 3.002 g of Ludox® AS-40 were added. The mixture was further stirred for 10 min at room temperature at 500 rpm before the addition of 0.046 g of CHA seed crystals prepared according to Reference Example 1. The mixture was charged to a 23 mL Teflon-lined autoclave. The tightly closed autoclave was placed in an oven pre-heated at 200° C. Hydrothermal treatment was carried out at 200° C. with 60 rpm tumbling for 20 h. Samples were collected using centrifugation at 14,000 rpm and washed with water until the pH of the washing water was in the range of from 7-8. The solid products were dried at 80° C. and calcined in air at 600° C. for 5 h.

The SEM of the material obtained after a synthesis time of 48 h is shown in FIG. 9. The XRD of the obtained materials after 3 h, 5 h, 16 h and 20 h is shown in FIG. 10. The crystallinity, determined as described in Reference Example 2.3, was only 7.4% after a crystallization time of 3 h and 19.4% after a crystallization time of 5 h (bottom curves). A reasonable crystallinity of about 100% was obtained only after a crystallization time of 16 h and 20 h (middle curve and top curve).

### Comparative Example 4: Process With Aging at 95° C. Using Aluminum Hydroxide as Source of Al

282.2 g of CHTMAOH (20 weight-% aqueous solution) and 82.4 g of TMAOH (25 weight-% aqueous solution) were first mixed. Then, 11.2 g of aluminum hydroxide (gibbsite) were added slowly under stirring. After stirring the mixture overnight at room temperature at 450 rpm, 300.4 g of Ludox® AS-40 were added. The mixture was further stirred for 20 min at room temperature at 450 rpm before the addition of 12 g of CHA seed crystals prepared according to Reference Example 1, milled and dried as described in Example 1. The mixture in the vessel was then stirred for 24 h at room temperature and the heated in a pre-heated oil bath to 95° C. and kept at this temperature under stirring at 450 rpm for 24 h. The thus heated and aged mixture (7.0 g) was then charged to a sealable tubular reactor made of stainless steel (type 1.4541) having an inner diameter of 12 mm, a tube length of 150 mm, and a tube wall thickness of 1.5 mm. The tightly sealed tube was placed in a pre-heated oven at 230° C. Hydrothermal treatment was carried out at 230° C. with 60 rpm tumbling for 1.5 and 2 h, respectively. Samples were collected using centrifugation at 14,000 rpm and washed with water until the pH of the washing water was in the range of from 7-8. The solid products were dried at 80° C. and calcined in air at 600° C. for 5 h.

It was found that after a crystallization time of 1.5 h, the crystallinity, determined as described in Reference Example 2.3, of the obtained zeolitic material having framework type CHA was only 32%, and only 30% after a crystallization time of 2 h.

### Comparative Example 5: Process With Aging at 95° C. Using Aluminum Triisopropylate as Source of Al

276.8 g of CHTMAOH (20 weight-% aqueous solution) and 78.0 g of TMAOH (25 weight-% aqueous solution) were first mixed. Then, 34.8 g of aluminum triisopropylate were added slowly under stirring. After stirring the mixture for 1.5 at room temperature at 450 rpm, 358.3 g of Ludox® AS-40 were added. The mixture was further stirred for 48 h at room temperature at 450 rpm before the addition of 5.3 g of CHA seed crystals prepared according to Reference Example 1. The mixture in the vessel was then stirred for 48 h at room temperature. The thus aged mixture was then charged to a sealable tubular reactor made of stainless steel (type 1.4541) having an inner diameter of 12 mm, a tube length of 150 mm, and a tube wall thickness of 1.5 mm. The tightly sealed tube was placed in a pre-heated oven at 210° C. Hydrothermal treatment was carried out at 210° C. with 60 rpm tumbling for 3 h, respectively. The sample was collected using centrifugation at 14,000 rpm and washed with water until the pH of the washing water was in the range of from 7-8. The solid products were dried at 80° C. and calcined in air at 600° C. for 5 h.

It was found that after a crystallization time of 3 h, the crystallinity, determined as described in Reference Example 2.3, of the obtained zeolitic material having framework type CHA was below 10 %.

### Comparative Example 6: Process With Aging at 95° C. Adding Seeds Before or After Aging

Four separate batch experiments were conducted to evaluate the effect of seeding before and after aging. All batches for aging were had a molar ratio of SiO₂/Al₂O₃/CHTMAOH/TMAOH/H₂O of 1/0.036/0.14/0.09/11.5 on the basis of a 20 mmol of SiO₂. Aqueous CHTMAOH (20 weight-% aqueous solution) and TMAOH (25 weight-% aqueous solution) were first mixed, followed by addition of Al(OH)₃ with stirring at 500 rpm for 30 minutes, followed by addition of Ludox®-40. Aging was conducted at 95° C. for duration of 1 or 4 days. Seeds were milled and dried according to reference example 1 and added either before or after the 1 or 4 day aging period. The aged batch including seeds was then divided into 4 equal portions and charged in 4 sealable tube reactors made of stainless steel (type 1.4541) having an inner diameter of 12 mm, a tube length of 150 mm, and a tube wall thickness of 1.5 mm. The tightly sealed tube was placed in a pre-heated oven at 200° C. Hydrothermal treatment was carried out at 200° C. with 60 rpm tumbling for 1, 2, 3 and 4 hour durations respectively. Samples were collected using centrifugation at 14,000 rpm and washed with water until the pH of the washing water was in the range of from 7-8. The solid products were dried at 80° C. and calcined in air at 600° C. for 5 h.

The crystallinity, determined as described in Reference Example 2.3 for times of 1 to 4 hours are shown in Table 1 below.

TABLE 1
Effect of seeding before and after aging
at 95° C. at 1 and 4 day aging periods.

| | | Crystal- | Crystal- | Crystal- | Crystal- |
|---|---|---|---|---|---|
| Aging | Seeding | linity | linity | linity | linity |
| duration | Added* | at 1 hr | at 2 hr | at 3 hr | at 4 hr |
| 1 day | before | 0.0% | 10.9% | 94.9% | 100.7% |
| 1 day | after | 1.3% | 1.7% | 78.6% | 96.5% |
| 4 days | before | 0.0% | 4.5% | 83.8% | 99.5% |
| 4 days | after | 0.6% | 2.3% | 57.0% | 88.8% |

*The words “before” and “after” indicate whether seeds prepared according to reference example 1 were added to the pre-crystallization synthetic mixture “before” the aging at 95° C. thus being present for the aging or immediately thereafter and prior to the hydrothermal treatment was commenced thus being absent from the aging process but present for crystallization.

### Comparative Example 7: Comparison of Reactivity of 1-Adamantyltrimethyl-Ammoniumhydroxide Mediated Aging Process Compared to Aqueous N,N,N-Trimethylcyclohexylammonium Hydroxide Aging Process

The following experiments demonstrate the difference in reactivity between the more reactive 1-adamantyltrimethyl-ammoniumhydroxide mediated process and the present inventive process. The structure directing agent, 1-adamantyltrimethyl-ammoniumhydroxide (253.6 g, 25 weight-% aqueous solution) was mixed with aqueous sodium hydroxide (28,8 g, 50 weight % aqueous solution) and water (64.8 g). Aluminum hydroxide (9.3 g) was added and the mixture stirred at room temperature for 30 minutes followed by Ludox® (300.5 g, 30 weight-% aqueous solution) and stirred a further 30 minutes. Seeds prepared according to reference example 1 (11.1 grams) were washed into the mixture with 540 g water. The mixture was stirred at 85° C. for 48 hours. After aging, the crystallinity of the aged mixture was analyzed according to reference example 2.3 and found to be 33% crystalline CHA type zeolite.

In comparison, aqueous N,N,N-trimethylcyclohexylammonium hydroxide (795.4 g, 20 weight-% aqueous solution) and TMAOH (163.3 g, 25 weight-% aqueous solution) were first mixed, followed by addition of Al(OH)₃ (78 g) with stirring at 500 rpm for 30 minutes, followed by addition of Ludox® (300.5 g, 30 weight-% aqueous solution). Seeds prepared according to reference example 1 (30.0 g) were added into the mixture. Aging was conducted at 82° C. for duration of 46 hours. After aging, the crystallinity of the aged mixture was analyzed according to reference example 2.3 and found to be amorphous.

### Example 1: Milling the Seed Material

The zeolitic seed material prepared as described in Reference Example 1 was milled using a bead-milling apparatus (LMZ015, Ashizawa Finetech Ltd.). 10 g of the zeolitic powder prepared according to Reference Example 1 were dispersed in 300 mL of water and milled with the bead-milling apparatus for 120 min at 3,000 rpm using zirconia beads with a diameter of 300 micrometer. In the vessel, 75% of the volume was filled with zirconia beads. The final concentration of the slurry was 7 weight-%. After the milling treatment, the slurry was optionally dried by centrifugation, and the residual solid was recovered. FIG. 1 shows XRD of the zeolitic seed material respectively prepared, and FIG. 2 shows the volume based particle size distribution (Dv) of the obtained seed material, and FIG. 3 and FIG. 4 show SEM images of the material.

### Example 2: Preparing a Zeolitic Material Having Framework Type CHA With Aging Using Aluminum Hydroxide as Source of Al and Dried Milled Zeolitic Seed Material in a Tubular Reactor

1.994 g of aqueous CHTMAOH solution (20 weight-%) and 0.669 g of aqueous TMAOH solution (25 weight-%) were first mixed. Then, 0.112 g of aluminum hydroxide (gibbsite) were added slowly under stirring. After stirring the mixture for 30 min at room temperature at 500 rpm, 3.008 g of Ludox® AS-40 were added. The mixture was further stirred at room temperature for 10 min at 500 rpm before the addition of 0.047 g of CHA seed crystals milled and dried according to Example 1. The mixture was aged at 65° C. for 2 days under stirring at 500 rpm. The aged mixture was divided into four portions and added to four tubular reactors made of stainless steel (type 1.4541) having an inner diameter of 12 mm, a tube length of 150 mm, and a tube wall thickness of 1.5 mm. Hydrothermal treatment was performed at 200° C. by placing the tubular reactors statically in an oven pre-heated at 200° C. The weights of the mixture in each tubular reactor were 1.327, 1.321, 1.354, and 1.388 g for hydrothermal treatments of 1, 2, 3, and 4 h, respectively. Samples were collected using centrifugation at 14,000 rpm and washed with water until the pH of the washing water was in the range of from 7-8. The solid products were dried at 80° C. and calcined in air at 600° C. for 5 h.

The SEM of the materials obtained after a synthesis time of 3 h is shown in FIG. 11. The XRD of the obtained materials after 1 h, 2 h, 3 h and 4 h is shown in FIG. 12. The crystallinity, determined as described in Reference Example 2.3, was already 23.4% after a crystallization time of 2 h (second curve from bottom) and 97% after a crystallization time of only 3 h (second curve from top). From a crystallization of 3 h to 4 h, the crystallinity increased to 98% (top curve).

### Example 3: Preparing a Zeolitic Material Having Framework Type CHA With Aging Using Aluminum Hydroxide as Source of Al and Dried Milled Zeolitic Seed Material in a Tubular Reactor

1.996 g of aqueous CHTMAOH solution (20 weight-%) and 0.663 g of aqueous TMAOH solution (25 weight-%) were first mixed. Then, 0.111 g of aluminum hydroxide (gibbsite) were added slowly under stirring. After stirring the mixture at room temperature for 30 min at 500 rpm, 3.007 g of Ludox® AS-40 were added. The mixture was further stirred for 10 min at 500 rpm at room temperature before the addition of 0.120 g of CHA seed crystals. The mixture was aged at 65° C. for 2 days while stirring at 500 rpm. The aged mixture was divided into four portions and added to four tubular reactors made of stainless steel (type 1.4541) having an inner diameter of 12 mm, a tube length of 150 mm, and a tube wall thickness of 1.5 mm. Hydrothermal treatment was performed at 200° C. by placing the tubular reactors statically in an oven pre-heated at 200° C. The weights of the mixture in each tubular reactor were 1.366, 1.390, 1.382, and 1.394 g for hydrothermal treatments of 1, 2, 3, and 4 h, respectively. Samples were collected using centrifugation at 14,000 rpm and washed with water until the pH of the washing water was in the range of from 7-8. The solid products were dried at 80° C. and calcined in air at 600° C. for 5 h.

The SEM of the materials obtained after a synthesis time of 3 h is shown in FIG. 13. The XRD of the obtained materials after 1 h, 2 h, 3 h and 4 h is shown in FIG. 14. The crystallinity, determined as described in Reference Example 2.3, was already 44.6% after a crystallization time of 2 h (second curve from bottom) and 96% after a crystallization time of only 3 h (second curve from top). From a crystallization of 3 h to 4 h, the crystallinity increased to 100% (top curve).

### Example 4: Preparing a Zeolitic Material Having Framework Type CHA With Aging Using Aluminum Hydroxide as Source of Al and a Zeolitic Seed Material Slurry in an Autoclave

3.989 g of an aqueous CHTMAOH solution (20 weight-%) and 1.313 g of an aqueous TMAOH solution (25 weight-%) were first mixed. Then, 0.225 g of aluminum hydroxide (gibbsite) were added slowly under stirring. After stirring the mixture for 30 min at room temperature at 500 rpm, 6.006 g of Ludox® AS-40 were added. The mixture was further stirred for 10 min before the addition of 1.316 g of a slurry (7 weight-%) of 120-min bead-milled CHA seed crystals, prepared as described above. The mixture was aged at 65° C. for 2 days while stirring at 500 rpm. The aged mixture was divided into two portions (6.431 and 6.126 g) and added to two 23-ml Teflon-lined autoclaves. The tightly closed autoclaves were placed in an oven pre-heated at 200° C. Hydrothermal treatment was carried out at 200° C. with 60 rpm tumbling for 5 h. Samples were collected using centrifugation at 14,000 rpm and washed with water until the pH became about 7-8. The solid product was dried at 80° C. and calcined at 600° C. wherein, for calcination, the solid product was heated from room temperature to 150° C. within 1 h, kept at 150° C. for 2 h, heated to a temperature of 600° C. within 5 h and kept at 600° C. for 5 h.

Elemental analysis showed a Si content of 38 weight-%, calculated as element, and an Al content of 2.8 weight-%, calculated as element. The SEM of the materials obtained after a synthesis time of 5 h is shown in FIG. 15. The XRD of the obtained materials after 5 h is shown in FIG. 16 for 6 different samples prepared according to this recipe. The average crystallinity, determined as described in Reference Example 2.3, was 95%.

### Example 5: Preparing a Zeolitic Material Having Framework Type CHA With Aging Using Aluminum Hydroxide as Source of Al and Dried Milled Zeolitic Seed Material in a Tubular Reactor

2.510 g of an aqueous CHTMAOH solution (20 weight-%) and 0.836 g of an aqueous TMAOH solution (25 weight-%) were first mixed. Then, 0.112 g of aluminum hydroxide (gibbsite) were added slowly under stirring. After stirring the mixture at room temperature for 30 min at 500 rpm, 3.004 g of Ludox® AS-40 was added. The mixture was further stirred for 10 min at room temperature before the addition of 0.119 g of dried and 120 min bead-milled CHA seed crystals, prepared as described above. The mixture was aged at 65° C. for 1 d while stirring at 500 rpm. The aged mixture was added to three tubular reactors made of stainless steel (type 1.4541) having an inner diameter of 12 mm, a tube length of 150 mm, and a tube wall thickness of 1.5 mm. Hydrothermal treatment was performed at 220° C. by placing the tubular reactors statically in an oven pre-heated at 220° C. The weights of the mixture in each tubular reactor were 1.535, 1.381, and 1.543 g for hydrothermal treatments of 1, 2, and 3 h, respectively. Samples were collected using centrifugation at 14000 rpm and washed with water until the pH of the washing water was in the range of 7-8. The solid product was dried at 80° C. and calcined at 600° C.

The SEM of the materials obtained after a synthesis time of 2 h is shown in FIG. 17. The XRD of the obtained materials after 1 h, 2 h, and 3 h is shown in FIG. 18. The crystallinity, determined as described in Reference Example 2.3, was already 66.8% after a crystallization time of 1 h (bottomcurve) and 95% after a crystallization time of only 2 h (middle curve).

### Example 6: Preparing a Zeolitic Material Having Framework Type CHA With Aging Using Aluminum Hydroxide as Source of Al and Dried Milled Zeolitic Seed Material in a Tubular Reactor

3.768 g of an aqueous CHTMAOH solution (20 weight-%) and 1.244 g of an aqueous TMAOH solution (25 weight-%) were first mixed. Then, 0.169 g of aluminum hydroxide (gibbsite) was added slowly under stirring. After stirring the mixture at room temperature for 30 min at 500 rpm, 4.506 g of Ludox® AS-40 were added. The mixture was further stirred for 10 min at 500 rpm at room temperature before the addition of 0.181 g of dried, 120-min bead-milled CHA seed crystals prepared as described above. The mixture was aged at 65° C. for 1 d while stirring at 500 rpm. The aged mixture was divided into six portions and added to six tubular reactors, made of stainless steel (type 1.4541) having an inner diameter of 12 mm, a tube length of 150 mm, and a tube wall thickness of 1.5 mm. Hydrothermal treatment was performed at 220° C. for 2 h by placing the tubular reactors statically in an oven pre-heated at 220° C. Samples were collected using centrifugation at 14,000 rpm and washed with water until the pH of the washing water was in the range of 7-8. The solid product was dried at 80° C. in air. The above procedure was repeated twice (i.e., 18 tubular reactors in total). The collected product was calcined at 600° C. for 5 h under dried air.

The molar silica:alumina ratio of the material was 26:1. The BET specific surface area was 635 m²/g. The SEM of the calcined material is shown in FIG. 19. The crystallinity of the obtained material was 97% after the crystallization time of only 2 h.

The ²⁷Al solid-state NMR spectrum of the material is shown in FIG. 20. The spectrum shows a main resonance at 59 ppm with a full width at half height of 4 ppm, which can be assigned to tetrahedrally coordinated Al. A resonance of less intensity than the main resonance, and not fully resolved from the main resonance, was observed at 30 ppm, which may be assigned to tetrahedrally or penta-coordinated Al. Another minor resonance of less intensity than the main resonance was observed at −3 ppm, which can be assigned to octahedrally coordinated Al. A spinning side band of the main resonance was observed at −35 ppm.

The ²⁹Si solid-state NMR spectrum of the material is shown in FIG. 21. The spectrum shows a resonance at −111 ppm with a full width at half height of 1.8 ppm, which we assign to Si(4 OSi, 0 OAl, 0 OH). The spectrum shows a second resonance, at −105 ppm, with a full width at half height of 2.8 ppm, which we assign to Si(3 OSi, 1 OAl, 0 OH). A further, unresolved resonance is observed within a range from −97 to −103 ppm, which may stem from Si(2 OSi, 2 OAl, 0 OH) or Si(3 OSi, 0 OAl, 1 OH).

### Example 7: Catalytic Testing Based on the Zeolitic Material Prepared According to Example 4

The zeolitic material prepared according the Example 4 above, in its dried and non-calcined state, was wet impregnated with an aqueous copper nitrate solution (incipient wetness impregnation). The material was then dried and calcined at 450° C. for 5 h. The respectively obtained zeolitic material having framework type CHA contained Cu, calculated as CuO, in an amount of 3.5 weight-%, based on the total weight of the material. The material was then shaped by preparing an aqueous slurry to which zirconium acetate was added as binder material precursor (5 weight-% based on zeolitic material). The slurry was then shaped to a tablet, dried under stirring and calcined for 1 h at 550° C. The respectively obtained tablet was then crushed and sieved to a particle size in the range of from 250 to 500 micrometer. The catalyst was then aged for 50 h at 650° C. in 10% steam/air, and for 16 h at 800° C. in 10% steam/air. Standard SCR conditions were applied by subjecting the catalytic material to a gas stream (500 ppm NO, 500 ppm NH₃, 5% H₂O, 10% O₂, balance N₂) at a gas hourly space velocity of 80,000 h⁻¹, at temperatures of the gas stream of 200° C., 400° C., 575° C. (first run for degreening); and 175° C., 200° C., 225° C., 250° C., 300° C., 450° C., 550° C., 575° C. The amount of the catalytic material was adjusted to 120 mg per reactor; the material was diluted with corundum to about 1 ml volume. The space velocities simulated 1 mL of a coated catalyst. The results of the simulated SCR tests are shown in FIGS. 22 and 23.

### BRIEF DESCRIPTION OF THE FIGURES

FIG. 1 shows XRD of the zeolitic seed material prepared according to Example 1.

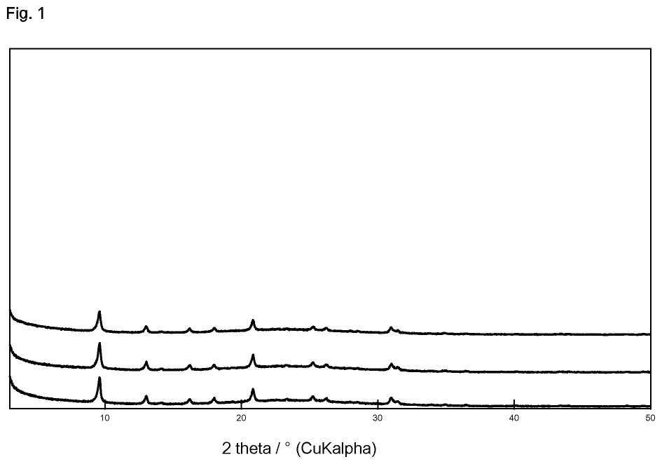

FIG. 2 shows particle size distribution of the seed material prepared according to Example 1.

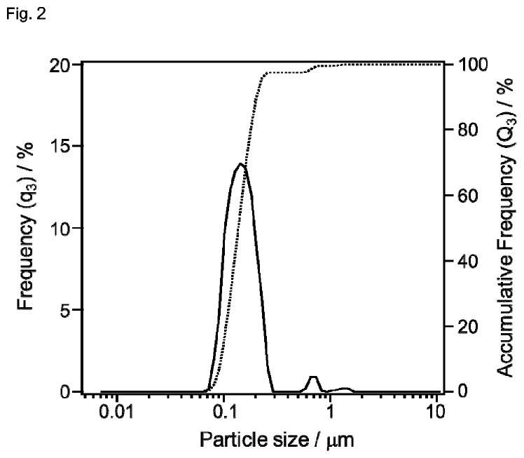

FIG. 3 shows SEM, prepared according to Reference Example 2.4, of the seed material prepared according to Example 1 (magnification 50,000).

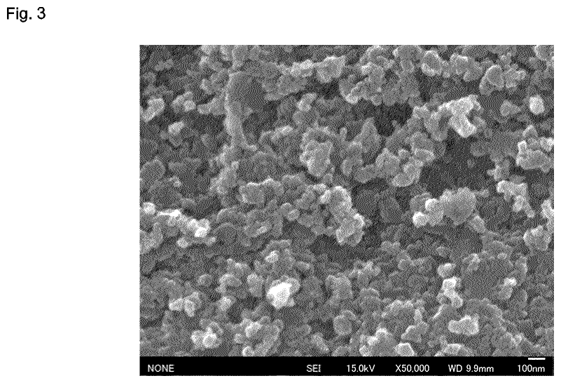

FIG. 4 shows SEM, prepared according to Reference Example 2.4, of the seed material prepared according to Example 1 (magnification 75,000).

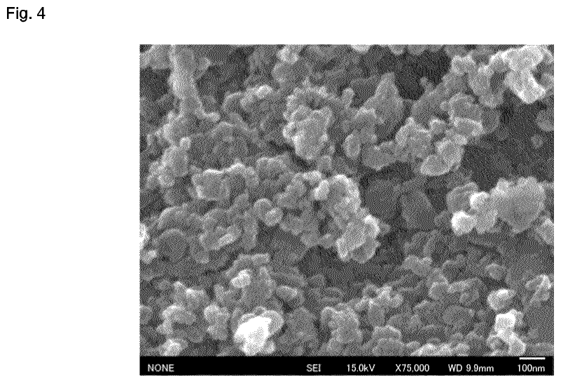

FIG. 5 shows the SEM, prepared according to Reference Example 2.4, of the zeolitic material prepared according to Comparative Example 1 (magnification 30,000).

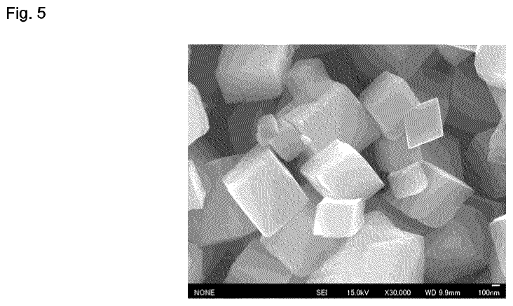

FIG. 6 shows the XRD plots of the zeolitic materials prepared according to Comparative Example 1. From bottom to top: crystallization time=20 h, 24 h, 48 h.

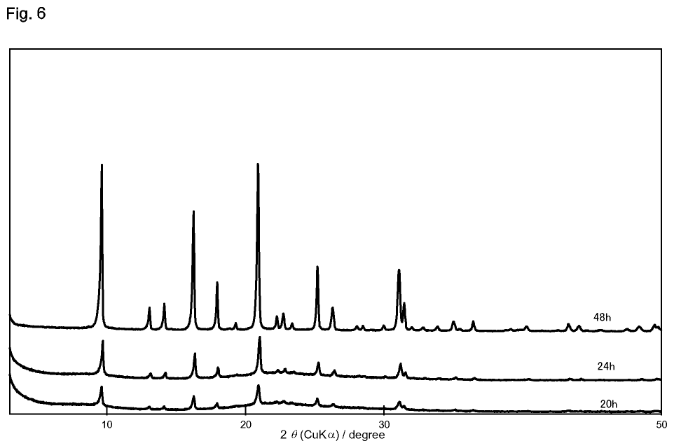

FIG. 7 shows the SEM, prepared according to Reference Example 2.4, of the zeolitic material prepared according to Comparative Example 2 (magnification 25,000).

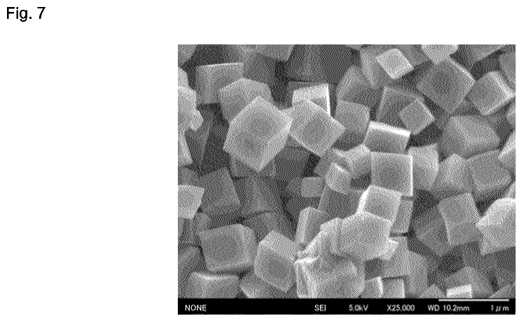

FIG. 8 shows the XRD plots of the zeolitic materials prepared according to Comparative Example 2. From bottom to top: crystallization time=3 h, 5 h, 16 h, 20 h.

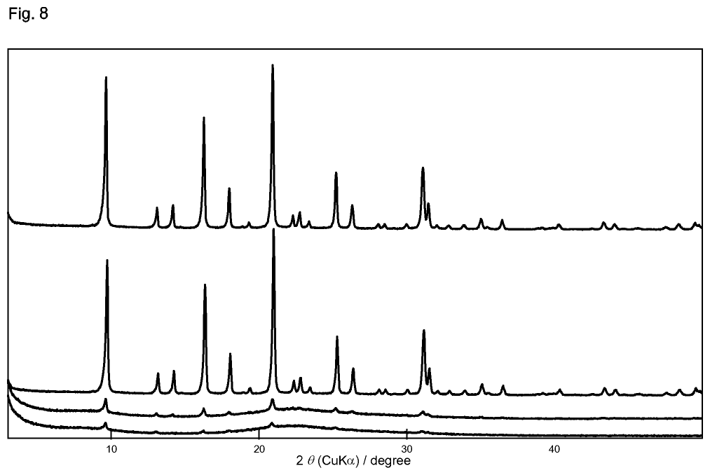

FIG. 9 shows the SEM, prepared according to Reference Example 2.4, of the zeolitic material prepared according to Comparative Example 3 (magnification 25,000).

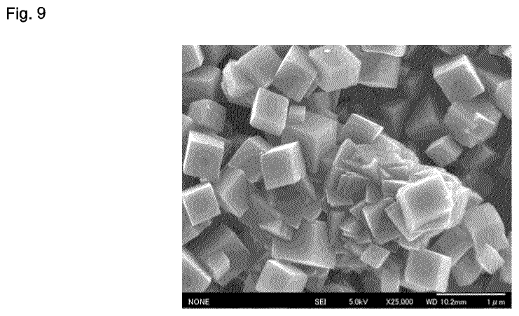

FIG. 10 shows the XRD plots of the zeolitic materials prepared according to Comparative Example 3. From bottom to top: crystallization time=3 h, 5 h, 16 h, 20 h.

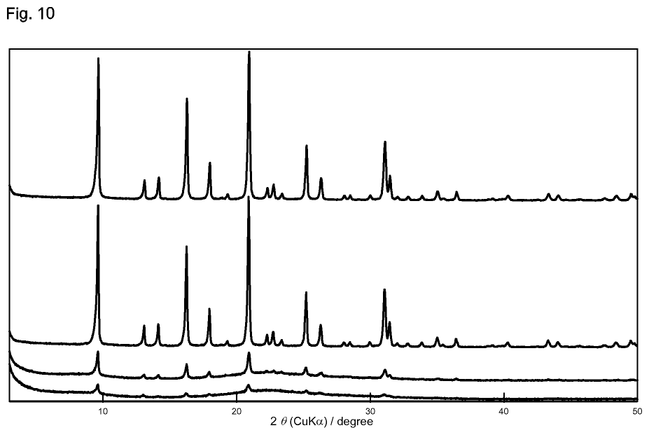

FIG. 11 shows the SEM, prepared according to Reference Example 2.4, of the zeolitic material prepared according to Example 2 (magnification 75,000).

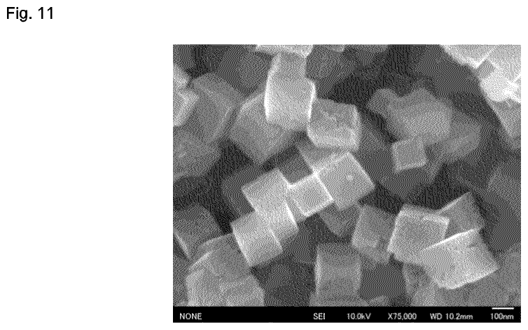

FIG. 12 shows the XRD plots of the zeolitic materials prepared according to Example 2. From bottom to top: crystallization time=1 h, 2 h, 3 h, 4 h.

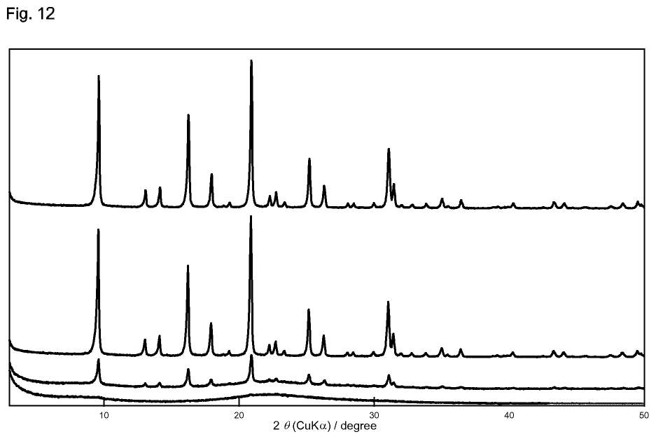

FIG. 13 shows the SEM, prepared according to Reference Example 2.4, of the zeolitic material prepared according to Example 3 (magnification 75,000).

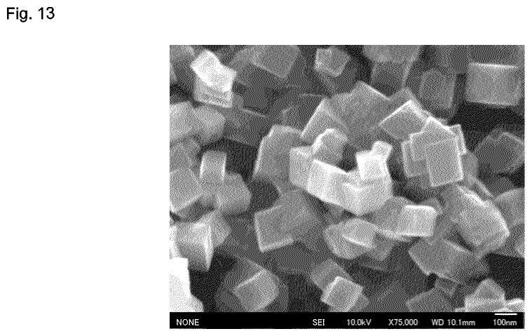

FIG. 14 shows the XRD plots of the zeolitic materials prepared according to Example 3. From bottom to top: crystallization time=1 h, 2 h, 3 h, 4 h.

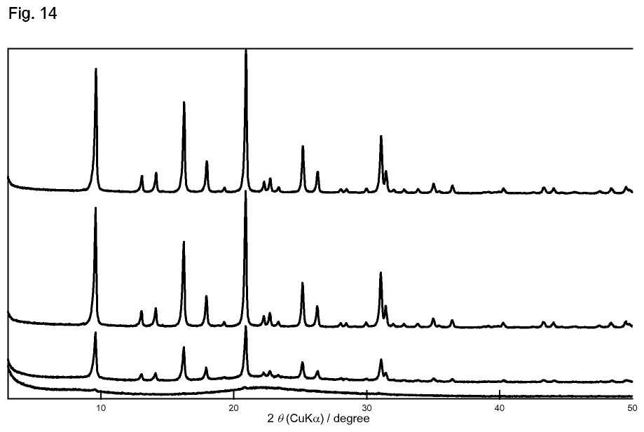

FIG. 15 shows the SEM, prepared according to Reference Example 2.4, of the zeolitic material prepared according to Example 4 (magnification 75,000 upper left, 50,000 upper right, 25,000 lower left, 10,000 lower right).

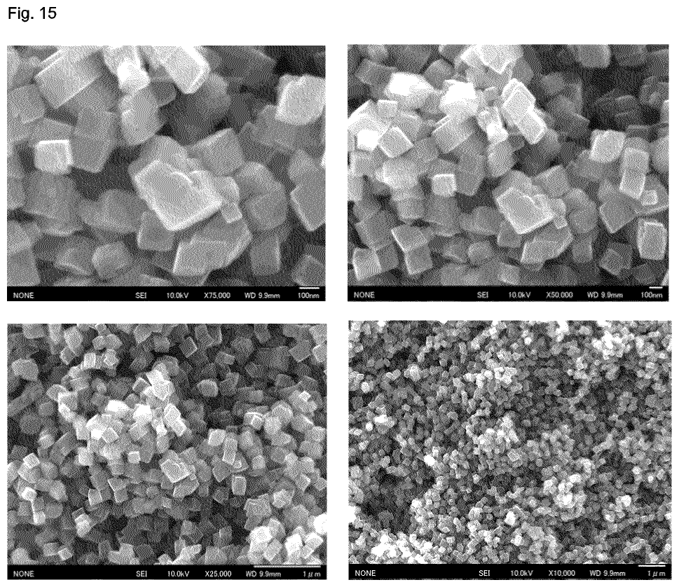

FIG. 16 shows the XRD plots of 6 samples of the zeolitic materials prepared according to Example 4, at a crystallization time of 5 h.

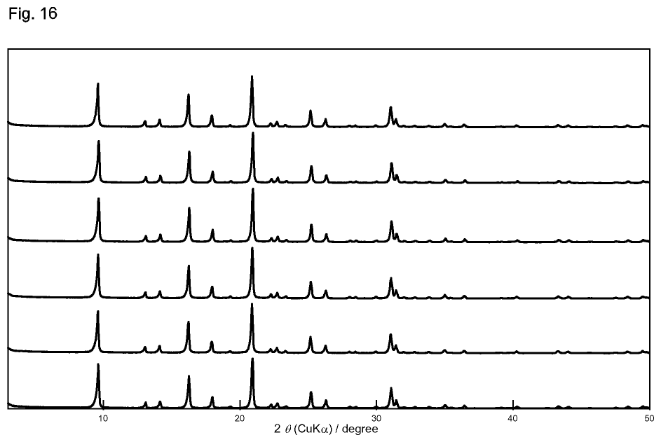

FIG. 17 shows the SEM, prepared according to Reference Example 2.4, of the zeolitic material prepared according to Example 5 after a crystallization time of 2 h (magnification 75,000).

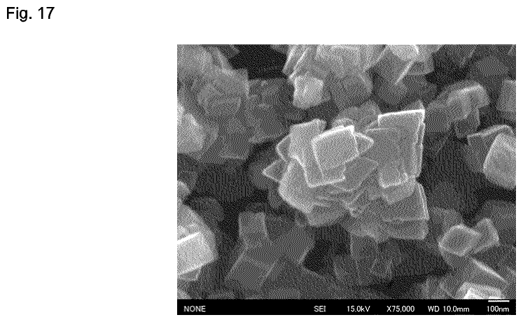

FIG. 18 shows the XRD plots of 3 samples of the zeolitic materials prepared according to Example 5, at a crystallization time, from bottom to top, of 1 h, 2 h, and 3 h.

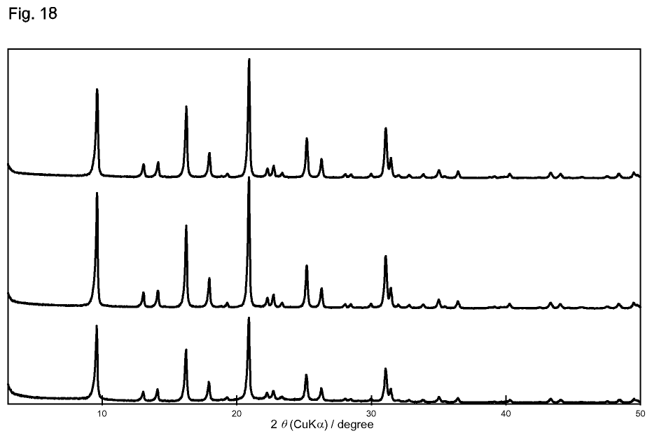

FIG. 19 shows the SEM, prepared according to Reference Example 2.4, of the zeolitic material prepared according to Example 6 (magnification 30,000).

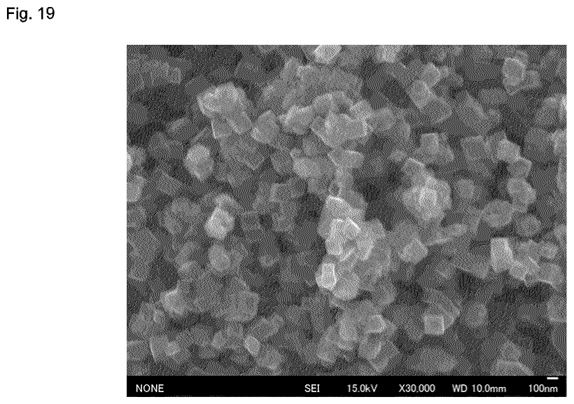

FIG. 20 shows the ²⁷AI solid-state NMR spectrum of the material prepared according to Example 6.

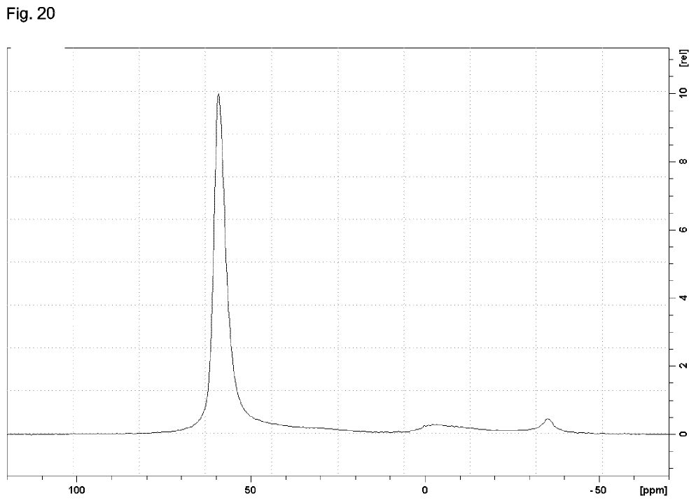

FIG. 21 shows the ²⁹Si solid-state NMR spectrum of the material prepared according to Example 6.

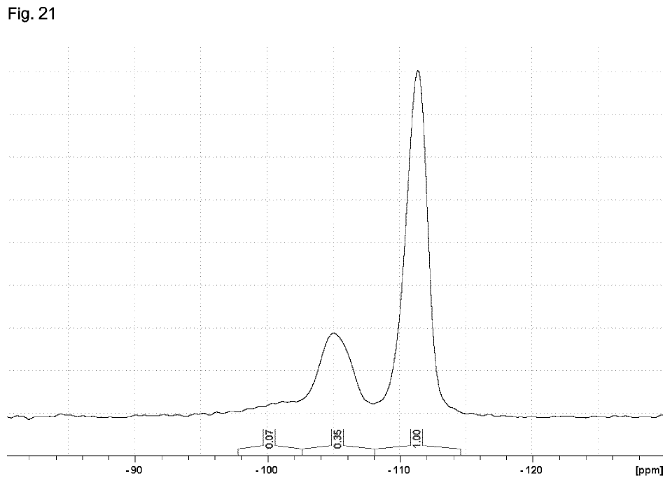

FIG. 22 shows the results of the catalytic testings according to Example 7 wherein the y axis shows X—NOx/%, wherein the curve with triangles down (7) shows the behavior of the catalytic material aged for 16 h, 800° C., run 2, the curve with triangles up (A) shows the behavior of the catalytic material aged for 50 h, 650° C., run 2, and the curve with circles show the behavior of a fresh standard SCR material.

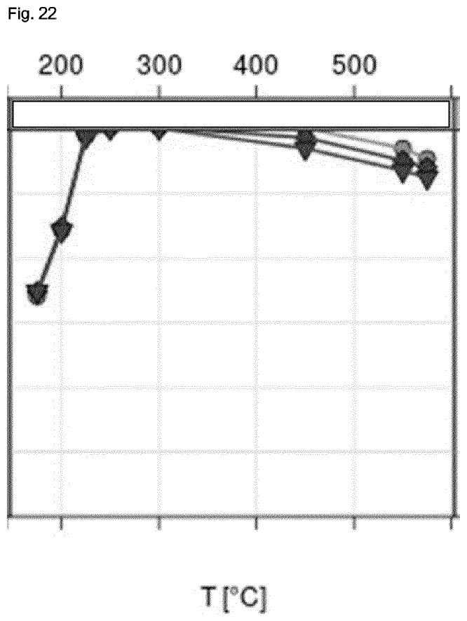

FIG. 23 shows the results of the catalytic testings according to Example 7 wherein the y axis shows N₂O/ppm, wherein the curve with triangles up (A) shows the behavior of the catalytic material aged for 16 h, 800° C., the curve with squares shows the behavior of the catalytic material aged for 50 h, 650° C. , and the curve with circles show the behavior of the fresh material.

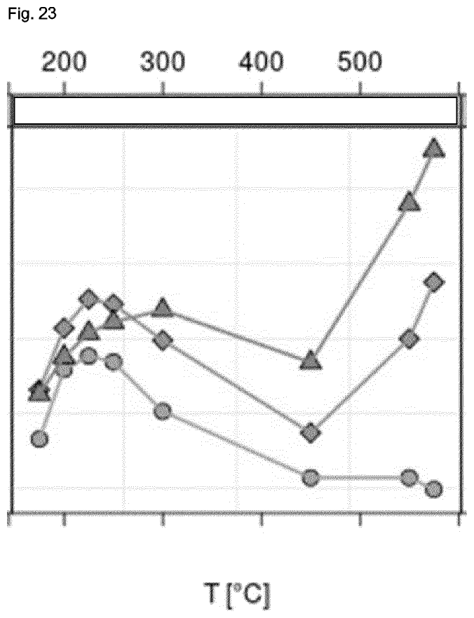

### CITED PRIOR ART

U.S. Pat. No. 4,544,538

WO 2015/185625 A

US 20170113941 A

1. A process for preparing a zeolitic material having a framework type CHA and a framework structure comprising Si, Al, O, and H, the process comprising:
providing a seeding material comprising a zeolitic material having a framework type CHA and a framework structure comprising Si, Al, O, and H;
(ii) preparing a mixture comprising a source of Si, a source of Al, the seeding material provided in (i), a CHA framework structure directing agent comprising a cycloalkylammonium compound, and water,
wherein the cycloalkylammonium compound is a compound comprising comprises a cation R¹R²R³R⁴N⁺,
wherein R¹, R², R³ are, each independently from one another, an alkyl residue having from 1 to 6 carbon atoms,
wherein R⁴ is a 5- to 8-membered cycloalkyl residue,
wherein in the mixture, a molar ratio of water relative to Si comprised in the source of Si and in the seeding material, calculated as SiO₂, is in a range of from 5:1 to 15:1,
wherein the mixture further comprises sodium and in the mixture, a molar ratio of sodium, calculated as Na₂O, relative to Si comprised in the source of Si and in the seeding material, calculated as SiO₂, is in a range of from 0:1 to 0.1:1;
(iii) heating the mixture prepared in (ii) in its liquid state to a temperature of the mixture in a range of from 50 to 90° C. and keeping the mixture at a temperature in this range for 5 to 100 I_{L} forming a heated mixture; and
(iv) heating the heated mixture of (iii) to a temperature of the heated mixture in a range of from 190 to 230° C. in a crystallization vessel and keeping the heated mixture at a temperature in this range under autogenous pressure in the crystallization vessel for 0.5 to 10 h, obtaining a solid material comprising the zeolitic material having a framework type CHA and a framework structure comprising Si, Al, O, and H, the solid material suspended in a mother liquor.

2. The process of claim 1, wherein providing the seeding material according to (i) comprises preparing the seeding material by a method comprising
(i.1) providing a zeolitic material having a framework type CHA and a framework structure comprising Si, Al, O, and H;
(i.2) preparing a suspension comprising the zeolitic material provided in (i.1) and a liquid; and
(i.3) milling the suspension prepared in (i.2).

3. The process of claim 2, wherein providing the seeding material according to (i) further comprises, after (i.3),
(i.4) separating the zeolitic material of the milled suspension obtained from (i.3) from the liquid, obtaining a separated zeolitic material,
wherein the separating comprises subjecting the milled suspension obtained from (i.3) to a solid-liquid separation, and
optionally drying the separated zeolitic material in gas atmosphere having a temperature in a range of from 20 to 100° C., wherein the gas atmosphere comprises oxygen and/or nitrogen.

4. The process of claim 1, wherein in the mixture prepared in (ii):
a weight ratio of the seeding material, relative to the Si comprised in the source of Si, calculated as SiO₂, is in a range of from 0.025:1 to 0.15:1,
a molar ratio of the CHA framework structure directing agent relative to Si comprised in the source of Si and in the seeding material, calculated as SiO₂, is in a range of from 0.20:1 to 0.30:1, and
a molar ratio of water relative to Si comprised in the source of Si and in the seeding material, calculated as SiO₂, is in a range of from 7:1 to 15:1.

5. The process of claim 1, wherein R¹, R², R³ are, each independently from one another, an alkyl residue having from 1 to 5 carbon atoms,
wherein R⁴ is a 5- to 7-membered cycloalkyl residue,
wherein the cycloalkylammonium compound comprises a hydroxide, and
wherein the CHA framework structure directing agent according to (ii) further comprises a tetraalkylammonium compound comprising a cation R⁵R⁶R⁷R⁸N⁺,
wherein R⁵, R⁶, R⁷, R⁸ are, each independently from one another, an optionally substituted alkyl residue having from 1 to 4 carbon atoms, and
wherein the tetraalkylammonium compound comprises a hydroxide.

6. The process of claim 1,
wherein in the mixture prepared in (ii), a molar ratio of the CHA framework structure directing agent relative to Si comprised in the source of Si and in the seeding material, calculated as SiO₂, is in a range of from 0.20:1 to 0.30:1.

7. The process of claim 1,
wherein the source of Si comprises at least one selected from the group consisting of a silica, a silicate, a fumed silica, a silica sol, an amorphous silica, a silica gel, a silicic acid, a silic acid ester, a colloidal silica, a tetraalkoxysilane, a disilicate, a sesquisilcate, and a silica hydrosol,
wherein the source of Al comprises at least one selected from the group consisting of an alumina, an aluminate, an aluminum salt, a tri(C1-C5)alkoxide, an AlO(OH), an Al(OH)₃, an aluminum halide, an aluminum fluoride, an aluminum chloride, an aluminum bromide, an aluminum sulfate, an aluminum phosphate, an aluminum fluorosilicate, a crystalline Al(OH)₃, and a gibbsite, and
wherein in the mixture prepared in (ii), a molar ratio of the source of Al, calculated as Al₂O₃, relative to Si comprised in the source of Si and in the seeding material, calculated as SiO₂, is in a range of from 0.001:1 to 0.5:1.

8. The process of claim 1, wherein preparing the mixture according to (ii) comprises
(ii.1) preparing a precursor mixture comprising the source of Si, the source of Al, the CHA framework structure directing agent comprising a cycloalkylammonium compound, and water,
wherein (ii1.) comprises the steps of
(ii.1.1) preparing a mixture comprising the source of Al and the CHA framework structure directing agent comprising a cycloalkylammonium compound;
(ii.1.2) agitating the mixture prepared in (ii.1.1) at a temperature of the mixture in a range of from 10 to 50° C. for 5 to 60 min;
(ii.1.3) adding the source of Si to the mixture obtained from (ii.1.2); and
(ii.1.4) agitating the mixture prepared in (ii.1.3) at a temperature of the mixture in a range of from 10 to 50° C. for 1 to 30 min, obtaining the precursor mixture; and
(ii.2) adding the seeding material to the precursor mixture prepared in (ii.1), obtaining the mixture to be subjected to (iii).

9. The process of claim 1, wherein according to (iii), the mixture prepared in (ii) is heated in its liquid state to a temperature of the mixture in a range of from 55 to 80° C., and kept at the temperature for 5 to 80 h, wherein during the keeping the mixture at the temperature the mixture is agitated.

10. The process of claim 1, wherein according to (iv), the heated mixture of (iii) is heated to a temperature of the heated mixture in a range of from 195 to 225° C., and kept at the temperature for 0.75 to 7.5 h.

11. The process of claim 1, further comprising:
(v) cooling the suspension of the solid material and the mother liquor obtained from (iv);
(vi) separating the solid material comprising a zeolitic material having a framework type CHA and a framework structure comprising Si, Al, O, and H, obtained from (v) from the mother liquor, obtaining a solid material comprising the zeolitic material having a framework type CHA and a framework structure comprising Si, Al, O, and H, the separating comprising
(vi.1) subjecting the suspension obtained from (v) to solid-liquid separation, obtaining the mother liquor and a solid material comprising the zeolitic material having a framework type CHA and a framework structure comprising Si, Al, O, and H;
(vi.2) washing the solid material obtained from (vi.1), obtaining a solid material comprising the zeolitic material having a framework type CHA and a framework structure comprising Si, Al, O, and H; and
(vi.3) drying the solid material obtained from (vi.1) and/or (v.2), obtaining a solid material comprising the zeolitic material having a framework type CHA and a framework structure comprising Si, Al, O, and H;
(vii) calcining the solid material obtained from (vi), obtaining a zeolitic material having a framework type CHA and a framework structure comprising Si, Al, O, and H; and
(viii) optionally cooling the zeolitic material obtained from (vii).

12. The process of claim 11, further comprising:
(ix) subjecting the zeolitic material having a framework type CHA and a framework structure comprising Si, Al, O, and H obtained from (vi), (vii), and/or (viii) to an ion-exchange process, obtaining a mixture comprising one or more cations M and a zeolitic material having a framework type CHA;
wherein according to (ix), the zeolitic material comprises one or more ionic non-framework elements that are ion-exchanged against one or more cations M, and
wherein the one or more cations M are selected from the group consisting of Sr, Zr, Cr, Mg, Mo, Fe, Co, Ni, Cu, Zn, Ru, Rh, Pd, Ag, Os, Ir, and Pt.

13. A zeolitic material having a framework type CHA and a framework structure comprising Si, Al, O, and H, obtained by the process according to claim 1.

14. A zeolitic material having a framework type CHA and a framework structure comprising Si, Al, O, and H,
wherein in the framework structure of the zeolitic material, a molar ratio of aluminum relative to silicon, calculated as a molar ratio of Al₂O₃:SiO₂, is in a range of from 0.001:1 to 0.5:1,
wherein:
the zeolitic material is in the form of crystals having a crystal size, determined via SEM, in a range of from 50 to 1,500 nm, wherein at least 50% of the crystals have a size in this range;
the zeolitic material has a BET specific surface area of at least 500 m²/g;
the zeolitic material has a ²⁷Al solid NMR spectrum exhibiting resonances and a peak maximum in a range of from 62.0 to 54.0 ppm with a full width at half height of at most 7.0 ppm;
the zeolitic material has a ²⁹Si solid NMR spectrum exhibiting
resonances and a peak maximum in a first range of from −108.1 to −114.5 ppm;
resonances and a peak maximum in a second range of from −102.6 to −108.1 ppm; and
a resonance with or without a peak maximum in a third range of from −97.7 to −102.6 ppm; and
a ratio of an integral according to the second range to an integral according to the first range is in a range of from 0.25:1 to 0.45:1.

15. A method for selective catalytic reduction of nitrogen oxides in an exhaust gas stream, the method comprising:
contacting the exhaust gas stream with a zeolitic material according to claim 14,
wherein the zeolitic material is used as at least one selected from the group consisting of an adsorbent, an absorbent, a molecular sieve, a catalytically active material, a catalyst, and a catalyst component.

16. A method for converting a Cl compound to one or more olefins, the method comprising:
contacting the Cl compound with a zeolitic material according to claim 14,
wherein the zeolitic material is used as at least one selected from the group consisting of an adsorbent, an absorbent, a molecular sieve, a catalytically active material, a catalyst, and a catalyst component.

17. A method for converting a synthetic gas comprising carbon monoxide and hydrogen to one or more olefins, the method comprising:
contacting the synthetic gas with a zeolitic material according to claim 14,
wherein the zeolitic material is used as at least one selected from the group consisting of an adsorbent, an absorbent, a molecular sieve, a catalytically active material, a catalyst, and a catalyst component.
